(19) United States
(12) Patent Application Publication
(10) Pub. No.: US 2023/0293881 A1
(43) Pub. Date: Sep. 21, 2023

(54) NEUROMODULATION FOR THE TREATMENT OF CIRCULATORY SYSTEM DISEASES

(71) Applicants: Aliaksandr GOURINE, Solihull (GB); Afferent Medical Solutions Ltd., Solihull (GB)

(72) Inventors: Aliaksandr Gourine, Solihull (GB); Andrey Gourine, Solihull (GB)

(21) Appl. No.: 18/017,527

(22) PCT Filed: Jul. 23, 2021
(86) PCT No.: PCT/EP2021/070755

§ 371 Date: Jan. 23, 2023

(30) Foreign Application Priority Data
Jul. 24, 2020 (GB) 2011522.6

(51) Int. Cl. A61N 1/04; A61N 1/36; A61B 5/021

(52) U.S. Cl. CPC A61N 1/0456; A61N 1/36034; A61N 1/36036; A61N 1/36031; A61N 1/0472; A61B 5/021; A61N 1/36017; A61B 5/02405

(57) ABSTRACT

A device and method are provided for modulating cerebral blood flow of a user by stimulating the nerves of a user. Modulating cerebral blood flow can be applied to the user for any of the following purposes: reducing systemic arterial blood pressure, reducing left ventricular myocardial hypertrophy, reducing pulmonary arterial blood pressure, treating heart failure and/or treating atrial fibrillation. An electrode is configured to be placed in contact with and provide the electrical stimulation signal to the nerves innervating a tragus of the user.

### RELATED APPLICATIONS

This application claims priority from Application PCT/EP2021/070755, filed Jul. 23, 2021, and claims priority from Great Britain Patent Application No. 2011522.6 filed Jul. 24, 2020, each of which are incorporated by reference in their entireties in this application.

### FIELD OF THE INVENTION

The present invention relates to a device and a method of non-invasive neuromodulation by application of a specific programme of electrical stimulation signals to cutaneous sensory projections of cranial nerves to modulate and improve blood flow in the brain (referred to herein as cerebral blood flow), subsequently leading to a reduction in the arterial blood pressure and left ventricular hypertrophy in human subjects with arterial hypertension or improvement of cardiac function in human subjects with heart failure. The present invention can be applied to the user for any of the following purposes: reducing systemic arterial blood pressure, reducing left ventricular hypertrophy, reducing pulmonary arterial blood pressure, treating heart failure and/or treating atrial fibrillation.

### BACKGROUND OF THE INVENTION

Systemic arterial hypertension, commonly referred to as “hypertension”, “essential hypertension” or “high blood pressure”, is a medical condition in which the systemic arterial blood pressure is chronically elevated.

Left ventricular hypertrophy (LVH) is a condition in which there is an increase in left ventricular myocardial mass, either due to an increase in wall thickness or due to left ventricular cavity enlargement, or both. Hypertension is the most common cause of LVH.

Pulmonary arterial hypertension is a medical condition in which the blood pressure in the pulmonary artery is chronically elevated.

Heart failure, also known as “congestive heart failure”, “congestive cardiac failure” or “chronic heart failure”, is a medical condition when the heart is unable to pump sufficiently to maintain adequate blood flow in the organs and tissues to meet the body's needs.

Atrial Fibrillation (referred herein to as AF) is a medical condition in which the patient experiences an abnormal heart rhythm (arrhythmia) characterized by rapid and irregular beating of the atrial chambers of the heart.

Hypertension is the leading health risk factor globally. High blood pressure is associated with adverse cardiovascular outcomes with elevated risk of myocardial infarction, heart failure, arterial aneurysms, kidney failure and stroke. Managing high blood pressure is critical: every 10 mmHg reduction in blood pressure results in a 17% reduction in coronary heart disease, 27% reduction in the incidents of stroke, 28% reduction in heart failure and 13% reduction in all-cause mortality. American Heart Association guidelines define hypertension as systolic blood pressure of 130 mmHg or greater or diastolic blood pressure of 80 mmHg or greater. Adoption of these guidelines labels 70.1 million people in the US and 15 million people in the UK in the 45-75 year age group as having hypertension, representing >60% of the population in this age group (J Am Coll Cardiol 71:e127, 2018). In Europe, it is estimated that only one third of hypertensive patients are diagnosed and treated to achieve the recommended levels of arterial blood pressure (Circulation 2016; 134:441-450). When hypertension is secondary to another medical condition, it is generally prudent to treat that primary condition first. A number of pharmacological therapies are available to treat high blood pressure. However, pharmacological treatments are often not effective; ˜15% of all hypertensive patients are drug-resistant (Hypertension. 2018; 72: e53-e90), most require taking two or more drugs, their efficacy is low in ˜50% of all patients, many patients need two or more drugs to control their blood pressure with >90% of these patients failing to control it within the recommended range with poor compliance. Underlying reasons include limited access to treatments, inadequate dosing or combination of treatments, poor patient adherence to treatment, the use of other interfering drugs, or the presence of treatment-resistant hypertension. A large number of patients with hypertension are also reluctant to adhere to pharmacological treatment regimens, because some medicines interfere with their daily lives, produce side effects, the patients prefer alternative medications, or for other reasons (BMJ 2012; 345:e3953).

Left ventricular hypertrophy (LVH) is present in 15% to 20% of the general population. It is more often prevalent in the elderly, the obese, and in patients with hypertension. Two-third of the patients with LVH are hypertensive. A review of echocardiographic data of 37,700 individuals revealed 19-48% prevalence of LVH in untreated hypertensive patients and 58-77%—in high-risk hypertensive patients (J Hum Hypertens 26: 343-349, 2012). LVH is a compensatory but ultimately, an abnormal increase in the mass of the myocardium of the left ventricle induced by a chronically elevated workload of the heart muscle. LVH diagnosis is based on the assessment of left ventricular mass. An echocardiogram is the test of choice in diagnosis of LVH.

Cardiac ultrasound utilizes transthoracic or transesophageal positioning of the transducer to measure the left ventricular end-diastolic diameter, posterior wall thickness, and interventricular septum thickness. From these measurements and the patient's height and weight, the left ventricular mass index can be calculated. LVH treatment should be aggressive because patients with LVH are at the highest risk of cardiovascular morbidity and mortality. The goal of therapy is to reduce LVH and prevent left ventricular dysfunction and progression to heart failure. The antihypertensive therapy benefits the patient by reducing arterial blood pressure and may reduce the degree of LVH, independently of blood pressure reduction, leading to a reduction of adverse cardiovascular events and mortality (Eur Heart J 39: 3021-3104, 2018).

Pulmonary hypertension encompasses a group of severe clinical entities, such as pulmonary arterial hypertension (PAH) in which the progressive loss and/or obstructive remodelling of the pulmonary vascular bed is responsible for the rise in pulmonary arterial pressure and pulmonary vascular resistance, resulting in a progressive right heart failure and right heart functional decline. Pulmonary hypertension is classified into five groups, depending on the cause. Group 1: Idiopathic PAH with unknown causes; Group 2: Pulmonary hypertension caused by left-sided heart disease; Group 3: Pulmonary hypertension caused by lung disease; Group 4: Pulmonary hypertension caused by chronic blood clots; and Group 5: Pulmonary hypertension associated with other conditions. Because current PAH treatments do not specifically target pulmonary vascular remodelling and inflammation, there is an urgent unmet clinical need to better identify the pathological mechanisms underlying the progressive narrowing of the pulmonary arterial lumen and perivascular inflammation and the loss of vessels in order to support therapeutic innovation aimed at reversing these changes and regenerating normal pulmonary vessels (Eur Respir J 53: 1801887, 2019)

Chronic heart failure (CHF) is one of the most common causes of morbidity and mortality in the developed world. Heart failure is associated with a diverse range of complications, including lethal arrhythmias and death as a result of the disease progression. In addition, CHF can be the terminal condition of many diseases of the circulatory system, including hypertension, myocardial infarction (MI), valvular heart disease, and various cardiomyopathies. CHF diagnosis is based on the assessment of cardiac left ventricular ejection fraction (LVEF). Heart failure with normal LVEF (≥50%) is defined as CHF with preserved ejection fraction (HFpEF), and CHF with low LVEF (<40%) as HF with reduced ejection fraction (HFrEF). The goals of heart failure therapy are to improve patients' clinical status, functional capacity and quality of life, reduce hospitalization rates and reduce mortality. Established pharmacological treatment of HFrEF involving several drug classes (including beta-adrenoceptor antagonists (or β-blockers), diuretics, and inhibitors of renin-angiotensin-aldosterone system) improves symptoms and reduces mortality rates. Yet, drug therapy remains insufficient, as cardiac function continues to deteriorate over time and most patients have poor prognosis. Moreover, currently there is no treatment for HFpEF, representing an urgent unmet clinical need.

Atrial fibrillation (AF) is an abnormal heart rhythm (arrhythmia) characterized by rapid and irregular beating of the atrial chambers of the heart (referred to as episodes). It often begins as short periods of abnormal beating which become longer or continuous as diseases progresses. It may also start as other forms of arrhythmia such as atrial flutter that then progresses into AF. Often episodes of AF have no symptoms. Occasionally there may be heart palpitations, fainting, light-headedness, shortness of breath, or chest pain. The disease is associated with an increased risk of heart failure, dementia, and stroke. The repeated occurrence of episodes is sometimes referred to as the AF burden which may be defined as the duration of the longest AF episode, number of AF episodes, and/or the percentage of time the patient is in AF during a certain period of time. There are four main types of AF: paroxysmal, persistent, long-term persistent, and permanent AF. The type of AF depends on how often AF occurs and how the patient responds to treatment. A brief event of AF is known as paroxysm AF, this type of AF usually stops in less than 24 hours but may also last for up to a week. Paroxysmal AF can happen repeatedly. Persistent AF is a condition in which the abnormal heart rhythm lasts for more than a week. It may stop spontaneously, but in most cases will need treatment. With this condition, the abnormal heart rhythms last for more than a year without going away. Sometimes AF burden does not improve, even when patients have tried several times to restore the normal heart rhythm with medicines or other treatments.

Despite significant advances in medical research, the biological/physiological mechanisms underlying the development of arterial hypertension are not fully understood. One hypothesis suggests that high blood pressure develops as a compensatory condition when blood supply to the brain is reduced, for example as a result of increased resistance of cerebral vasculature. Blood flow in the brain (cerebral blood flow) is driven by the arterial blood pressure and inversely proportional to cerebrovascular resistance. Any increase in the resistance to blood flow, for example due to (cerebro)vascular disease, atherosclerotic legions, ageing, etc., would require compensatory increases in the arterial pressure to maintain brain perfusion, essential to support the function of brain nerve cells processing information. Therefore, it is possible to treat circulatory system disease (in general) and reduce systemic arterial blood pressure in patients with hypertension (in particular) by applying methods or treatments designed to improve blood flow in the brain.

Blood flow to the brain comes from two sources: Internal carotid arteries (supply the anterior brain) and vertebral arteries (supplying the brainstem and posterior brain) that maintain cerebral circulation (i.e. blood flow in the brain), which is the movement of blood through the dense network of cerebral arteries, capillaries and veins. The rate of the cerebral blood flow in the healthy adult human being is an average approximately 750 millilitres per minute but may be reduced in several disease states leading to compensatory responses as described above.

US2019351230 discloses an electrostimulation device includes a computer generating an electrostimulation generator control signal and outputting a music signal, a transcutaneous electrostimulation generator, an electronic signal conduit, and an electrode coupler. US2019351230 makes reference to a first electrode on a first face of the tragus and a second electrode on the second face of the tragus, however, these are used to provide a connection with a third electrode contacting the skin of the auditory canal. Disclosed current pathways in US2019351230 include pathways between either the first or the second electrode and the third electrode.

### SUMMARY OF THE INVENTION

The present invention describes a device and a method for non-invasive electrical stimulation of cranial nerves that project to the skin of the outer ear to modulate cerebral blood flow of a user. In some embodiments the electrical stimulation is for the purpose of modulating the function of the cardiovascular system of a user, such as modulating blood pressure or functional and electrical properties of the heart. One potential avenue for arterial blood pressure control is via modulation of the blood flow in the brain, and in particular in the brainstem—the area of the brain that controls the cardiovascular system. According to the present invention, the above purposes can be achieved non-invasively by stimulation (i.e. electrical) of cutaneous sensory projections of certain cranial and spinal nerves that originate from the brainstem and spinal cord. Activation of these nerves is expected to facilitate the blood flow through the lower brainstem leading to a reduction in the arterial blood pressure and associated cardiac work. Regions of the external ear, and the tragus in particular, are innervated by the sensory branches of the fifth (V) and the tenth (X) cranial nerves as well as branches of the spinal nerves C2 and C3. According to the present invention, global cerebral blood flow and blood flow through the brainstem in particular can be facilitated by transcutaneous or percutaneous electrical stimulation of the sensory nerves projecting to of the outer ear According to further aspect of the invention, electrical stimulation of these nerves can achieve a therapeutic effect in the treatment of circulatory system disease.

The present disclosure is related to the field of medical treatment of cardiovascular disease, including hypertension, or high arterial blood pressure, pulmonary arterial hypertension, heart failure and atrial fibrillation.

In a first aspect of the invention there is provided a device for modulating cerebral blood flow of a user. The device comprises a generator configured to produce an electrical stimulation signal and a controller, connected to the generator and configured to determine the form of the electrical stimulation signal. The device also includes an earpiece, connected to the generator and controller and the earpiece has an electrode (or a pair of electrodes). The controller transmits the electrical stimulation signal to the electrode(s) and the electrode(s) are configured to be placed in contact with and provide the electrical stimulation signal to the skin of a tragus of the user (such as across the tragus). The electrical stimulation signal comprises a series of electrical pulses, each pulse repeats with a frequency of 1 Hz to 100 Hz and each pulse has duration of 10 microseconds to 500 microseconds and an amplitude of 0.1 mA to 8 mA. The device is used by the user at least once a day for at least 3 consecutive days.

The device may be applied to the user to modulate cerebral blood flow and/or for any of the following purposes: reducing systemic arterial blood pressure, reducing left ventricular hypertrophy, reducing pulmonary arterial blood pressure, treating cardiovascular conditions such as heart failure and atrial fibrillation.

According to the embodiment, the device can be applied to the user such that the electrical stimulation signal is applied transcutaneously or percutaneously.

In some embodiments the earpiece is configured to apply the electrical stimulation signal transcutaneously. In such embodiment's electrodes comprise non-piercing conductive surfaces. In some embodiments the earpiece is configured to apply the electrical stimulation signal percutaneously. In such embodiments the electrodes comprise conductive surfaces configured to pierce the surface of the skin to make electrical contact with a subsurface layer of tissue.

The device can be applied to the user for a minimum of 5 minutes and a maximum of 2 hours per day.

The device may have a first and second electrode, the first electrode is configured to be placed in contact with the left tragus of the user and the second electrode is configured to be placed in contact with the right tragus of the user. Additionally, the device may have a first earpiece and a second earpiece, and the first earpiece has the first electrode and the second earpiece has the second electrode.

In some embodiments the first electrode is a first stimulating electrode and the second electrode is a second stimulating electrode. Each earpiece comprises a stimulating electrode and a reference electrode wherein the electrical stimulation signal is applied between the stimulating electrode and the reference electrode.

The device may comprise a first pair of electrodes and a second pair of electrodes, wherein the first pair of electrodes is configured to be placed in contact with the left tragus of the user and the second pair of electrodes is configured to be placed in contact with the right tragus of the user. The device may comprise a first earpiece and a second earpiece, wherein the first earpiece is configured to be placed in contact with the left tragus and comprises the first pair of electrodes and the second earpiece is configured to be placed in contact with the right tragus and comprises the second pair of electrodes. In each pair of electrodes, one electrode may be a stimulating electrode and one electrode may be a reference electrode.

In some embodiments the first and the second earpiece are substantially identical. In some embodiments the first earpiece is shaped to fit on the left tragus and the second earpiece is shaped to fit on the right tragus. In some embodiments the left and the right earpieces are substantially mirror images of each other. In some embodiments an earpiece is shaped to conform to a portion of the tragus or another part of the ear, such that the earpiece fits preferentially to the tragus such that the stimulating electrode is in contact with the outer side of the tragus and the reference electrode is in contact with the inner side of the tragus. In some embodiments an earpiece is shaped to conform to a portion of the tragus or another part of the ear, such that the earpiece fits preferentially to the tragus such that the stimulating electrode is in contact with the inner side of the tragus and the reference electrode is in contact with the outer side of the tragus.

The device may further include a securing means configured to secure the electrode to a tragus of a user. The securing means may include a clip and the clip may include a first gripping portion and a second gripping portion which are biased into contact with each other. The stimulating electrode may be located on the first gripping portion, when the gripping portion is present.

There may also be a reference electrode located on the first or second gripping portion.

The device may include a physiological sensor configured to measure the value of a physiological parameter (e.g. heart rate, blood pressure) and optionally may store the value in a memory portion of the device. The device may also include a temperature sensor configured to measure the temperature of an area of the skin of the user. The temperature sensor may be configured to measure the temperature of the tragus. The device may store data from the temperature sensor in a memory portion of the device. The values stored in the memory portion is used by the controller can be used to determine the form of the electrical stimulation signal. In some embodiments the physiological sensor and/or the temperature sensor may be provided on an earpiece, for example located on a clip forming part of the earpiece.

In some embodiments, the physiological sensor measurements, temperature sensor measurements and time and date information on the use of the device by the patient are recorded and stored in the memory portion of the device.

The device may comprise means to measure one or more of: the voltage applied between the stimulating electrode and the reference electrode; the current flowing between the stimulating electrode and the reference electrode; and the time relation between the voltage and the current.

The device may comprise a stimulating electrode, a counter electrode and/or a reference electrode, and may be configured to measure one or both of: the voltage between the stimulating electrode and the reference electrode, and the voltage between the counter electrode and a reference electrode. In this way, the potential of the stimulating, counter and/or the reference electrodes may be measured without error arising from voltage drop across the electrode to skin interface, as known in the art.

In some embodiments the device is configured to control the current flowing between the stimulating electrode and the reference electrode as a function of time, according to the form of the electrical stimulation signal, by means of a feedback control means using one or more measured values. In some embodiments the device is configured to control the voltage applied between the stimulating electrode and the reference electrode as a function of time, according to the form of the electrical stimulation signal, by means of a feedback control means using one or more measured values.

In some embodiments the device is configured to derive the phase relationship between the voltage between the stimulating electrode and the reference electrode and the current flowing through the stimulating electrode and the reference electrode, for example to measure the electrical impedance of the tragus.

Measurement of the electrical impedance between the stimulating electrode and the reference electrode may be used to determine one or more of: that the electrodes are correctly positioned on the opposing faces of the tragus; that the device and method of the invention is in use, and the times and for the duration that the device and method are used; and to control the voltage and/or current in response to the impedance, for example to compensate for variation in the structure or conductivity of the tragus.

Additionally, measurements of current, voltage and phase relationship of the electrical stimulation signal can be stored in a memory portion of the device and used to determine the electrical impedance of the tragus.

The controller can be configured to produce the electrical stimulation signal and the pattern of stimulation based on a user input received at the controller. This user input includes at least one of the following: pulse duration, waveform, pulse frequency, pulse pattern and current or voltage amplitude of the electrical stimulation signal.

Example features of the second and subsequent aspects of the invention are as for the first aspect mutatis mutandis.

In a second aspect of the invention there is provided a system for non-invasive electrical stimulation of nerves that project to the skin of the outer ear and, in some embodiments, for the purpose of modulating cerebral blood flow of a user. The system includes a device as disclosed herein and further comprises a communication module connected to the controller of the device. The communication module can be configured to send information from the device to an external computer system and to receive information from the external computer system, and the information received from the external computer system can be used by the controller to determine the form of the electrical stimulation signal.

The system is further configured to act upon information from the device received by the external computer system, wherein the information is compared to a secondary set of information stored on the external computer system to determine a set of actions to be performed by the device and/or the external computer.

In a third aspect of the invention there is provided a method of modulating cerebral blood flow of a user of the device. The method comprises producing, using a generator, an electrical stimulation signal; determining, using a controller connected to the generator, the form of the electrical stimulation signal; transmitting, the electrical stimulation signal to an electrode, for example to an electrode pair or pairs. The stimulating electrode is configured to be placed in contact with and provide the electrical stimulation signal to the skin of a tragus of the user. The electrical stimulation signal comprises a series of electrical pulses, each pulse repeats with a frequency of 1 Hz to 100 Hz and each pulse has a duration of 10 microseconds to 500 microseconds and an amplitude of 0.1 mA to 8 mA. The device is applied to the user at least once a day for at least 3 consecutive days.

In another aspect of the invention there is provided a method for non-invasive electrical stimulation of nerves that project to the skin of the outer ear using a device as disclosed herein, comprising the steps of: bringing the stimulating electrode and the reference electrode into contact with the tragus of a user; producing, using the device, an electrical stimulation signal applied to the stimulating electrode and the reference electrode; and determining, using the controller, the waveform and the frequency of the electrical stimulation signal, wherein the electrical stimulation signal comprises a series of electrical pulses, each pulse repeating with a frequency of about 1 Hz to about 100 Hz and each pulse has a duration of about 10 microseconds to about 500 microseconds and an amplitude of about 0.1 mA to about 20 mA.

In some embodiments, the electrical stimulation signal comprises a series of electrical pulses, each pulse repeating with a frequency in the range about 3 Hz to about 50 Hz and each pulse having a duration of about 100 microseconds to about 500 microseconds and an amplitude of about 0.1 mA to about 8 mA.

In some embodiments the frequency is in the range from about 1 Hz to about 100 Hz, such as about 1 Hz to 10 Hz, 10 Hz to 20 Hz, 20 Hz to 30 Hz, 30 Hz to 40 Hz, 40 Hz to 50 Hz, 50 Hz to 60 Hz, 60 Hz to 70 Hz, 70 Hz to 80 Hz, 80 Hz to 90 Hz, or 90 Hz to about 100 Hz.

In some embodiments the frequency is in the range 3 Hz to 20 Hz, 5 Hz to 30 Hz, 10 Hz to 50 Hz, 15 Hz to 60 Hz, 20 Hz to 75 Hz, 25 Hz to 80 Hz, 30 Hz to 100 Hz.

In some embodiments the frequency is in the range 3 Hz to 50 Hz.

In some embodiments the pulse has a duration in the range about 10 microseconds to about 500 microseconds, such as about 10 microseconds to 100 microseconds, 20 microseconds to 200 microseconds, 30 microseconds to 300 microseconds, 40 microseconds to 400 microseconds, 50 microseconds to about 500 microseconds.

In some embodiments the pulse has a duration in the range 100 microseconds to 200 microseconds, 200 microseconds to 300 microseconds, 300 microseconds to 400 microseconds, 400 microseconds to 500 microseconds.

In some embodiments the pulse has a duration in the range 50 microseconds to 200 microseconds, 100 microseconds to 250 microseconds, 200 microseconds to 500 microseconds.

In some embodiments the pulse has a duration in the range 100 microseconds to 500 microseconds.

In some embodiments the amplitude is in the range about 0.1 mA to about 10 mA, such as about 0.1 mA to about 2 mA, about 0.2 mA to about 5 mA or about 0.5 mA to about 10 mA.

In some embodiments the amplitude is in the range 0.1 mA to 1 mA, 0.2 mA to 2 mA, 0.3 mA to 3 mA, 0.4 mA to 4 mA, 0.5 mA to 5 mA, 0.6 mA to 6 mA, 0.7 mA to 7 mA, 0.8 mA to 8 mA, 0.9 mA to 9 mA or 1.0 mA to 10 mA.

In some embodiments the amplitude is in the range 0.1 mA to 5 mA, 0.5 mA to 8 mA, 1 mA to 10 mA, or 2 mA to 20 mA.

In some embodiments the amplitude is in the range about 0.5 mA to about 5 mA.

In some embodiments the method is applied to the user to modulate cerebral blood flow, and/or for any of the following purposes: reducing systemic arterial blood pressure, reducing left ventricular hypertrophy, reducing pulmonary arterial blood pressure, treating cardiovascular conditions such as heart failure and atrial fibrillation

In a fourth aspect of the invention there is provided a method for the treatment of a disease or condition selected from the group consisting of hypertension (high blood pressure), left ventricular hypertrophy, heart failure, atrial fibrillation, comprising administering to a subject an electrical stimulation signal using the device and/or methods described herein. Additionally, the method may be applied for the treatment of a disease or condition of one or more of the group consisting of hypertension (high blood pressure), left ventricular hypertrophy, heart failure, atrial fibrillation at the same time. In some embodiments the method is applied to modulate, such as to increase, cerebral blood flow. The electrical stimulation signal comprises a series of electrical pulses, each pulse repeats with a frequency of 1 Hz to 100 Hz and each pulse has a duration of 10 microseconds to 500 microseconds and an amplitude of 0.1 mA to 8 mA. The device is applied to the user at least once a day for at least 3 consecutive day.

The stimulating and reference electrodes may be configured to be placed in contact with an outward facing surface and an inward facing surface of the tragus. In some embodiments, the electrical stimulation signal may be transmitted to at least a first pair of electrodes and a second pair of electrodes, wherein the first pair of electrodes is placed in contact with the skin of the left tragus of the user and the second pair of electrodes is placed in contact with the skin of right tragus of the user. The electrical stimulation signal applied to each of the left and right tragi may be substantially the same electrical stimulation signal and the signal may be applied simultaneously or sequentially to each of the left and right tragi. Alternatively, the electrical stimulation signal applied to the left tragus may be different from the electrical stimulation signal applied to the right tragus and the electrical stimulation signal applied to the left and right tragi may be applied simultaneously or sequentially to each of the left and right tragi. Alternatively, the electrical stimulation signal applied to the left tragus is different from the electrical stimulation signal applied to the right tragus and the electrical stimulation signal applied to the left tragus may be applied at a different time to the electrical stimulation signal applied to the right tragus.

When using the device and applying the method of treatment, the electrical stimulation signal may be of a sinusoidal, square, triangular, pulse or “white noise” waveform. The electrical stimulation signal may be a pulse waveform, the pulse being substantially a sinusoidal, square, triangular, or “white noise” waveform. The generated waveform can be monophasic symmetrical waveform, or a biphasic symmetrical waveform, or a triphasic symmetrical waveform. Alternatively, the generated waveform can be a biphasic asymmetrical waveform, or a triphasic asymmetrical waveform.

The method can be applied to the user for a minimum of 5 minutes and a maximum of 2 hours per day.

The device can be applied to the user for a minimum of 5 minutes and a maximum of 2 hours per day.

The method or device can be applied to the user for a minimum of 5 minutes and a maximum of 2 hours per day and the method is applied at intervals separated by at least one day.

The method or device can be applied to the user for a minimum of about 5 minutes and a maximum of about 2 hours per day and the method may be applied at intervals in the range about 1 day (i.e. 24 hr) to about 7 days, such as about 1 day to about 2 days.

In methods of treatment according to the invention, the method or device can be applied to the user for different periods. In an embodiment of a process of treatment according to the invention, during a first period, the method is applied to the user for between 5 minutes and 2 hours each day, the first period comprising a minimum of 3 consecutive days; during a second period the method is stopped for at least 2 days and during a third period the method is applied to the user for between 5 minutes and 2 hours each day.

In a fifth aspect the invention provides a method of treatment of a medical condition of a user comprising applying the device and method of the invention to achieve non-invasive electrical stimulation of nerves that project to the skin of the outer ear, in combination with providing a medication to the user. In some embodiments use of the device and/or method of the invention modulates the pharmacological effect of the medication.

The method of treatment according to the present invention may therefore further comprise a step of administering to the patient (i.e. the user of the device as described herein) a pharmaceutically active composition for the treatment of a disease or condition selected from the group consisting of hypertension, left ventricular hypertrophy, heart failure and/or atrial fibrillation, e.g. an agent to treat hypertension, heart failure and/or atrial fibrillation. Additionally, the method may be applied for the treatment of a disease or condition of one or more of the group consisting of hypertension (high blood pressure), left ventricular hypertrophy, heart failure, atrial fibrillation at the same time. Such pharmaceutically active compositions may be administered separately, sequentially or simultaneously with the use of the device as described herein.

Suitable anti-hypertensive compositions may comprise diuretics, beta-adrenoceptor antagonists (β-blockers), angiotensin converting enzyme inhibitors, angiotensin II receptor blockers, calcium channel blockers, alpha-adrenoceptor antagonists (alpha-blockers), alpha-2 receptor agonists, and/or combined alpha- and β-blockers. For example, suitable pharmaceutically active substances for use as anti-hypertensive agents include, but are not limited to, alfuzosin hydrochloride, ambrisentan, atenolol, bisoprolol, bosentan, clonidine hydrochloride, doxazosin, epoprostenol, furosemide, hydralazine hydrochloride; iloprost, indoramin, macitentan, methyldopa, metoprolol, minoxidil, moxonidine, prazosin, propranolol, riociguat, sildenafil, sodium nitroprusside, tadalafil, tamsulosin hydrochloride, terazosin.

Suitable compositions for the treatment of heart failure may comprise diuretics, beta-adrenoceptor antagonists (β-blockers), angiotensin converting enzyme inhibitors, angiotensin II receptor blockers, calcium channel blockers. Other medicines suitable for use in the treatment of heart failure include but are not limited to heart rate lowering agents (for example ivabradine), blood thinners (for example antiplatelet drugs or anticoagulant drugs). Suitable antiplatelet drugs include, but are not limited to, anagrelide, aspirin, clopidogrel, prasugrel, ticagrelor, tirofiban, vorapaxar, dipyridamole. Suitable anticoagulant drugs include, but are not limited to, dabigatran, edoxaban, rivaroxaban, apixaban, warfarin, enoxaparin, dalteparin, fondaparinux.

Suitable antiarrhythmic agents for the treatment of atrial fibrillation include, but are not limited to, beta-adrenoceptor antagonists (β-blockers) (for example acebutolol, atenolol, betaxolol, labetalol, bisoprolol, carvedilol, metoprolol tartrate, metoprolol succinate, nebivolol, penbutolol, propranolol, sotalol hydrochloride, timolol, nadolol, pindolol), calcium channel blockers (for example verapamil hydrochloride, diltiazem hydrochloride), digitalis glycosides (for example digoxin), sodium channel blockers (for example disopyramide, mexiletine, quinidine, procainamide, propafenone, flecainide), potassium channel blockers (for example amiodarone, dronedarone, sotalol).

The method may further comprise measuring the blood pressure of a user; determining whether the blood pressure of the user is greater than a predetermined threshold value; and if the blood pressure of the user is greater than the predetermined threshold value, instructing the device to produce the electrical stimulation signal.

The agent may be provided to the user at a dose selected to produce a therapeutic effect in combination with use of the method of the invention. In some embodiments the dose is selected to be within the range known in the art to achieve therapeutic effect in the condition. In some embodiment the dose is selected to be below the range conventionally used in the art to achieve therapeutic effect in the condition. In the latter embodiments of the method of treatment, use of the device and method of the invention combines with the pharmacological mode of action of the agent, such that the potency of the agent in the condition may be enhanced. In this way, a therapeutic effect results from the combination of the method of the invention and the agent, while any side effects arising from the agent are reduced as a result of the use of a lower dose.

The determining, using a controller connected to the generator, the form of the electrical stimulation signal pulse comprises determining the pulse duration, pulse frequency, waveform and waveform pattern of the electrical stimulation signal.

Furthermore, a method of identifying a patient suitable for treatment by a device and method of treatment of the present invention is disclosed. The method includes recording an electrocardiogram of a patient for a minimum period of 1 minute; analysing the power spectrum of heart rate variability; determining the low frequency (LF) to high frequency ratio (HF) of heart rate variability spectrum (LF/HF); and determining whether the LF/HF ratio of heart rate variability spectrum is greater than a predetermined threshold value. In some embodiments, the predetermined threshold value of LF/HF ratio heart rate variability spectrum is 1 or about 1.

Another method of identifying a patient suitable for treatment by a device and method of treatment of the present invention is also disclosed. The method includes recording the baseline value of the heart rate of a patient when resting in a supine position, followed by recording the heart rate of the patient when actively standing in a vertical position, wherein the heart rate is recorded directly after the patient stands in the vertical position and then subsequently after a period of time; recording the heart rate of the patient again while standing; and then determining whether the difference between the heart rate value recorded immediately after standing and heart rate value recorded subsequently is less than a predetermined value. The predetermined value of heart rate difference between the value recorded immediately after standing and heart rate value recorded subsequently while standing is suitably less than 6 (six) beats per minute.

Suitably, the patient is supine for 5 to 15 minutes, optionally 5 to 10 minutes, in an example, about 10 minutes. The patient suitably actively stands in the vertical position in about 5 to 10 seconds, suitably in 5 seconds. The recording of the heart rate of the patient while standing can be for around 1 to 5 minutes, suitably 1 to 3 minutes, in an example, 1 minute. The recording of the heart rate subsequently while standing can be made between 10 to 30 seconds after standing, suitably 10 to 20 seconds after standing.

In one embodiment, the method can comprise recording the supine baseline values of heart rate for 1 minute, whilst the patient is resting in the supine position for 10 minutes; the patient standing up, wherein the patient stands up in a time period of less than 5 seconds; recording the patient's heart rate continuously for 1 minute after standing up; calculating the difference between heart rate values obtained while standing and baseline supine values of heart rate; determining whether the difference between the heart rate value recorded immediately after standing and heart rate value recorded between seconds and 20 seconds after standing is less than a predetermined value. The predetermined threshold value of heart rate difference between the value recorded immediately after standing and heart rate value recorded between 10 seconds and 20 seconds after standing is less than 6 (six) beats per minute.

The methods of screening of the present invention may therefore further optionally comprise a step of administering to the patient a pharmaceutically active composition for treatment of a disease or condition selected from the group consisting of hypertension, heart failure and/or atrial fibrillation, e.g. an agent to treat hypertension, heart failure and/or atrial fibrillation, where the pharmaceutically active substance may be as described above.

### BRIEF DESCRIPTION OF THE DRAWINGS

Various embodiments of the invention are described by way of examples with references to the accompanying drawings in which:

FIG. 1A depicts sites on the left tragus and the right tragus of the human outer ear that receive sensory nerve innervation. Electrical stimulation of these nerves transcutaneously or percutaneously modulates cerebral brain blood and can be used to treat diseases of the circulatory system according to the present invention.

FIG. 1B depicts an embodiment of an earpiece comprising an electrode clip.

FIG. 1C illustrates an electrode clip

FIG. 1D illustrates positioning of an earpiece as depicted in FIG. 1B on the tragus.

FIG. 1E depicts a cross section through the tragus with the stimulating and reference electrodes in contact with the outer and inner surfaces of the tragus respectively, showing current flow pathways through the tragus.

FIG. 2 illustrates changes in cerebral blood flow induced by non-invasive neuromodulation, applied in an animal model using different parameters of electrical stimulation to the outer ear according to the present invention.

FIGS. 3A and 3B depict the blood pressure values in drug-resistant hypertensive patients before and after the use of non-invasive neuromodulation using a device and a method of treatment according to the invention.

FIGS. 4A and 4B depict the blood pressure values in uncontrolled hypertensive patients before and after the use of non-invasive neuromodulation using a device and a method of treatment according to the invention.

FIG. 4C depicts the values of left ventricular myocardial mass in uncontrolled hypertensive patients before and 12 months after the use of non-invasive neuromodulation using a device and a method of treatment according to the invention.

FIGS. 5A and 5B depict the blood pressure values in uncontrolled hypertensive patients before and after the use of non-invasive neuromodulation using a device and a method of treatment according to the invention, applied in combination with the beta-adrenoceptor antagonist bisoprolol.

FIG. 6 depicts a description and circuit block drawing for a device according to the invention.

FIGS. 7A and 7B depict possible waveforms of the electrical stimulation signal.

### DETAILED DESCRIPTION

The terms “subject”, “individual” and “patient” as used herein refer to humans, which do not denote a particular age or sex. In certain embodiments the individual subject may be a patient, a subject that is a candidate for, or awaiting medical or other treatment, such as the method of device-based neuromodulation described herein. The term “about” as used herein means in quantitative terms plus or minus 10%. For example, “about 5 mmHg” would encompass the range 4.5-5.5 mmHg.

### Hypertension

The disclosed device and method can be used to treat hypertension and lower systemic arterial blood pressure in a subject involving identifying a subject diagnosed with hypertension. The disclosed device and method can also be used to modulate cerebral blood flow for the purpose of lowering systemic arterial blood pressure in a subject involving identifying a subject diagnosed with hypertension. The term hypertension as used herein refers to a condition or disease well known in the art in which the systemic arterial blood pressure in a human subject is chronically elevated.

To prevent, diagnose, and treat hypertension, blood pressure is categorized as normal (less than 120 mmHg systolic and less than 80 mmHg diastolic), elevated (120 to 129 mmHg systolic and less than 80 mmHg diastolic), stage 1 hypertension (130 to 139 mmHg systolic or 80 to 89 mmHg diastolic), or stage 2 hypertension (more than 140 mmHg systolic or more than 90 mmHg diastolic). Patients whose systolic and diastolic blood pressures are in different categories are assigned to the higher stage (for example a patient with a blood pressure of 128/82 mmHg should be diagnosed with stage 1 hypertension).

Hypertension may refer to a condition in which a subject's resting systolic arterial blood pressure is above 120 mmHg and/or diastolic arterial blood pressure is above 80 mmHg. In certain embodiments hypertension may refer to a condition in which a subject's resting systolic arterial blood pressure is above any of the following limits: about 115 mmHg, about 120 mmHg, about 125 mmHg, about 130 mmHg, about 135 mmHg, about 140 mmHg, about 145 mmHg, about 150 mmHg, about 155 mmHg, about 160 mmHg, about 165 mmHg, about 170 mmHg and/or when the systemic diastolic arterial blood pressure is above any of the following limits: about 80 mmHg, about 85 mmHg, about 90 mmHg, about 95 mmHg, about 100 mmHg, about 105 mmHg, about 110 mmHg, about 115 mmHg, about 120 mmHg. In some embodiments, systemic arterial hypertension may be chronic treatment-resistant hypertension, defined as sustained arterial blood pressure level above the recommended target (24 h ambulatory systolic blood pressure higher than 130 mmHg) despite documented treatment with at least three antihypertensive medications in adequate doses, one of which is a diuretic. Diagnosis of hypertension in a subject may in various embodiments be performed by an individual qualified to make such diagnosis in a particular jurisdiction.

Left ventricular hypertrophy is diagnosed in patients if the left ventricular myocardial mass indexed to body surface area (LVMI) is greater than 95 g/m² for women and greater than 115 g/m² for men.

### Pulmonary Arterial Hypertension

The disclosed device and method can also be used to treat pulmonary hypertension. Pulmonary arterial hypertension may refer to a condition in which a subject's resting pulmonary systolic arterial blood pressure is above about 25 mmHg.

### Heart Failure

The disclosed device and method can also be used to treat heart failure. The terms heart failure, or congestive heart failure, or chronic heart failure as used herein refer to a condition or disease well known in the art in which the heart is unable to pump sufficiently to maintain blood flow in the organs and tissues to meet the body's needs. In certain embodiments heart failure may refer to a condition in which a subject's left ventricular ejection fraction is above about 50% (HFpEF), or between about 40% and about 49% (heart failure with mid-range ejection fraction), or lower than about 40% (HFrEF).

### Atrial Fibrillation

The disclosed device and method can also be used to treat atrial fibrillation or AF. AF refers to a condition or disease well known in the art in which the normal regular electrical impulses generated by the sinoatrial node in the right atrium of the heart are overwhelmed by disorganized electrical impulses usually originating in the roots of the pulmonary veins. This leads to irregular conduction of electrical impulses that generate the heartbeat.

### Method and Device for Modulating the Cerebral Blood Flow

The present invention employs a device and a specific method of non-invasive neuromodulation to modulate cerebral blood flow via electrical stimulation of afferent (sensory) branches of cranial nerves innervating the tragus (e.g. the auricular region) and projecting to the brainstem, for the purpose of lowering arterial blood pressure as the medical treatment of hypertension and left ventricular hypertrophy, or reducing cardiac work to improve heart function as the medical treatment of heart failure, or reducing the number and frequency of AF episodes. Additionally, the device and method may be applied for the treatment of a disease or condition of one or more of the group consisting of hypertension (high blood pressure), left ventricular hypertrophy, heart failure, atrial fibrillation at the same time. For example, a patient with both hypertension and AF could be treated for both conditions simultaneously using the claimed method or device.

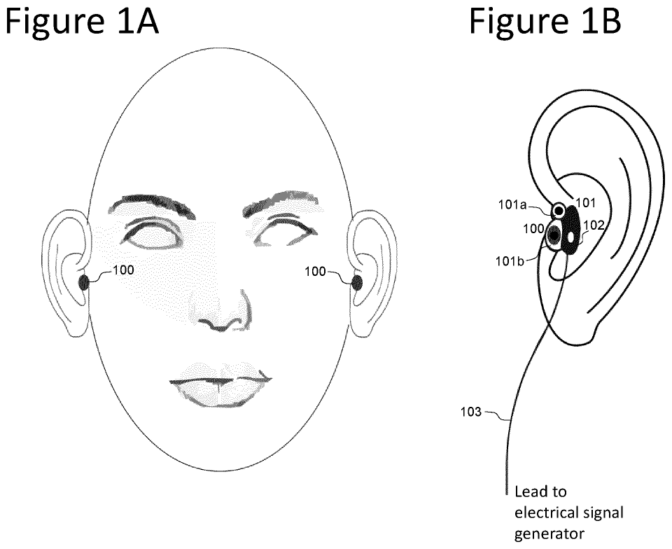

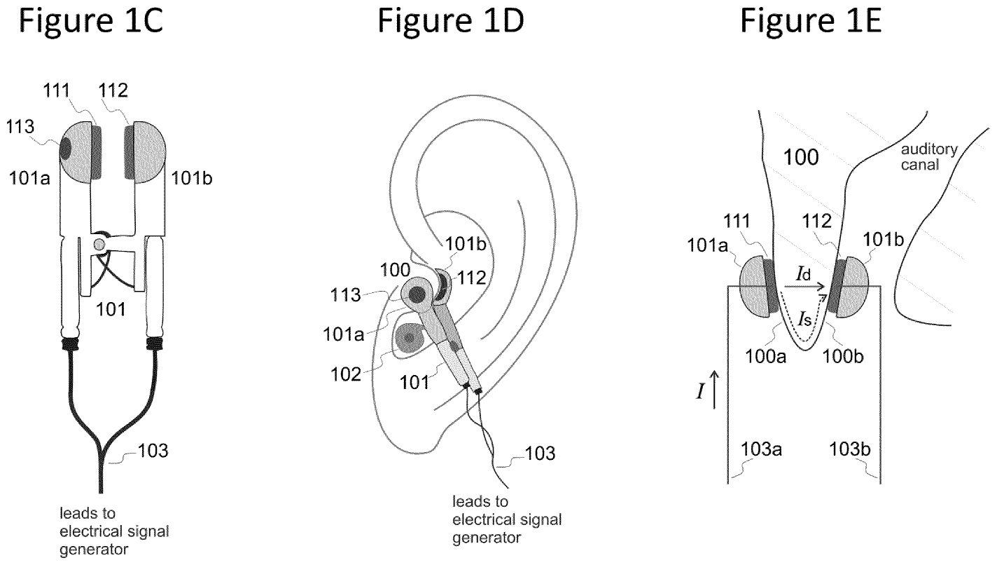

More specifically, the present invention achieves a reduction in blood pressure in hypertensive individuals, reduces left ventricular hypertrophy, reduces the AF burden, and improves cardiac function in heart failure patients by non-invasive neuromodulation, produced by a specific stimulation treatment programme involving delivery of electrical pulses with specific characteristics applied transcutaneously (to the skin) or percutaneously (using electrodes through the skin) to the inward and outward facing regions of the tragus of both ears (FIG. 1A). At its broadest, the present invention reduces blood pressure in hypertensive individuals, reduces left ventricular hypertrophy, reduces AF burden, and improves cardiac function in heart failure by stimulating cranial and spinal nerve fibers innervating the tragus region of the outer ear to modulate cerebral blood flow.

FIG. 1 illustrates the sites of electrical stimulation to activate nerves projecting to the skin of the tragus in order to modulate cerebral blood flow and treat diseases of the circulatory system.

FIG. 1 depicts a device for modulating cerebral blood flow of a user. The device comprises a generator configured to produce an electrical stimulation signal; a controller, connected to the generator and configured to determine the form of the electrical stimulation signal and an earpiece, connected to the generator and controller, the earpiece comprising a pair of electrodes (FIGS. 1B and 1C), e.g. a stimulating electrode and a reference electrode. The earpiece is connected to the generator and controller via a lead. Alternatively, the earpiece may be connected to the generator and controller via a wireless connection. In such embodiments, the earpiece comprises an earpiece signal generator for generating the electrical stimulation signal, a wireless receiver and a power supply, wherein the earpiece signal generator is configured to receive instructions from the wireless receiver and to apply the electrical stimulation signal to the stimulating electrode and the reference electrode.

The controller is configured to produce the electrical stimulation signal and the pattern of stimulation based on a user input received at the controller. The controller can therefore adjust the parameters of the electrical stimulation signal depending on the required treatment plan for the user. The controller may be connected to a communication module to deliver information to the controller from an external source. Alternatively, the controller can be controlled by the user of the device directly. The user input includes at least one of the pulse duration, waveform, pulse frequency, pulse pattern and current amplitude of the electrical stimulation signal. The user input may also include information on the duration of usage of the device and interval period between using the device for subsequent rounds of treatment. For example, informing the user that the device to be used for a period of between 5 min and 2 hours each day for a minimum of 3 consecutive days.

FIG. 1A depicts the schematic depiction of a human head and depicts the tragus 100 on each ear. The regions of particular interest for the present invention are the left tragus and the right tragus 100.

FIG. 1B depicts the placement of the earpiece on the tragus of a user. FIG. 1C illustrates an embodiment of an earpiece in the form of an electrode clip. FIG. 1D depicts the placement of the earpiece shown in FIG. 1C onto the tragus of a user. The same pairs of electrodes can be applied to each of the left tragus and the right tragus, known as bilateral stimulation (i.e. to both the left and right tragi). Improved results are noted when using bilateral stimulation compared to using stimulation of just one tragus. A first earpiece may be placed on the left tragus and a second earpiece may be placed on the right tragus. In this way, pairs of stimulating and reference electrodes, are placed on each of the tragi. The stimulating electrode may be placed on the outer skin surface of the tragus and the reference electrode on the inner skin surface of the tragus. The device enables both the left and the right tragi to be electrically stimulated for the purpose of modulating the cerebral blood flow. The electrical stimulation signal generated by the device may be applied to each of the left or right tragi on their own or both simultaneously.

The electrical stimulation signal applied to the left and right tragi may be substantially the same electrical stimulation signal, i.e. the signal applied to the left and right tragi may have substantially the same waveform. Alternatively, the electrical stimulation signal applied to the left tragus may be different from the electrical stimulation signal applied to the right tragus (i.e. the signal applied to the left and right tragi may have different waveforms). The electrical stimulation signal applied to the left and right tragi may be applied simultaneously or sequentially to each of the left and right tragi. ‘Simultaneously’ means that the electrical stimulation signal is applied to the left and right tragus at substantially the same time. ‘Sequentially’ means that the electrical stimulation signal is first applied to one of the left or right tragus and is then subsequently applied to the opposite tragus. This action can be repeated several times to continuously apply the electrical stimulation signal to each of the left and right tragus in turn (for example, at 5 second intervals). Alternatively, the electrical stimulation signal can be applied to the left tragus at a different time from the electrical stimulation signal applied to the right tragus. ‘Different’ means that the electrical stimulation signal may be applied to only one of the left or right tragus and not both at the same time.

### Earpiece

In some embodiments, the earpiece is configured to bring the stimulating electrode into contact with the outer face of the tragus of a user and the reference electrode into contact with the inner face of the tragus of the user. In some embodiments, each earpiece comprises only two electrodes.

In some embodiments, the earpiece comprises only two current-carrying electrodes, and the stimulating electrode and the reference electrode are the current-carrying electrodes. In some embodiments the earpiece comprises a stimulating electrode, a counter electrode and a reference electrode, wherein the stimulating electrode and the counter electrode are the current-carrying electrodes, and the reference electrode is operable with the controller to determine the electrical stimulation signal. In some embodiments, the earpiece is configured to bring all three electrodes into contact with the tragus, for example, to bring the stimulating electrode and the reference electrode into contact with one face of the tragus and the counter electrode into contact with the other face. In some embodiments, the earpiece is configured to bring the stimulating electrode and the reference electrode into contact with the outer face of the tragus and the counter electrode into contact with the inner face. In some embodiments, the earpiece is configured to bring the stimulating electrode into contact with one face of the tragus and the reference electrode and the counter electrode into contact with the other face.

In some embodiments, the stimulating electrode and the reference electrode are provided on the earpiece such that when the stimulating electrode and the reference electrode are in contact with the tragus and the electrical stimulation signal is applied, the current flow between the stimulating electrode and the reference electrode is primarily between the outer and the inner surfaces of the tragus, through the tissue of the tragus.

In some embodiments the earpiece is configured such that the current flow between the stimulating electrode and the reference electrode is exclusively between the outer and the inner surfaces of the tragus, through the tissue of the tragus. In this way, in such embodiments the nerves innervating the surface, and/or the interior tissue of the tragus are electrically excited by the potential difference between the stimulating electrode and the reference electrode electrodes, and/or the current flowing through the tissue surrounding the nerves.

In some embodiments the electrical stimulation signal is selected such that over the course of a series of cyclically repeating pulses, there is a net conventional current flow from the stimulating electrode to the reference electrode. In some embodiments the net conventional current flow from the stimulating electrode to the reference electrode is positive. In other embodiments the net conventional current flow is negative.

In some embodiments the earpiece is configured to bring the stimulating electrode into contact with the outer face of the tragus of a user and the reference electrode into contact with the inner face of the tragus of the user; the electrical stimulation signal comprises a cyclically repeating series of pulses; and the electrical stimulation signal is selected such that during each cycle there is a net conventional current flow from the stimulating electrode to the reference electrode.

In some embodiments the stimulating electrode is the positive electrode and the reference electrode is the negative electrode and the net current flow is from the stimulating electrode to the reference electrode. In other embodiments the stimulating electrode is the negative electrode and the reference electrode is the positive electrode and the net current flow is from the reference electrode to the stimulating electrode.

In some embodiments the earpiece further includes a securing means to secure the electrodes to the tragus and hold them in place for an extended period of time such that the treatment can be continuously delivered to the user. The securing means is configured to secure the earpiece and the electrodes in place over the skin of the tragus. The securing means may include a clip or the earpiece itself may take the form of a clip, as illustrated by FIG. 1C. For example, in some embodiments, the clip may be configured to secure the earpiece electrodes in place by gripping a user's tragus, with a first gripping portion and a second gripping portion on respective sides of the tragus. Where this is the case, the stimulating electrode may be located on the first gripping portion, and a reference electrode may be located on the second gripping portion. Either the first gripping portion or the second gripping portion may extend into the ear canal. A physiological sensor may also be located on the clip and is in an example also located on the first gripping portion or the second gripping portion. Alternatively, the physiological sensor may be present on part of the device, such as on a part of the earpiece which is not the clip.

In example embodiments, the clip is shaped to provide an ergonomic fit on the tragus. This is advantageous for delivery of electrical pulses and for monitoring of physiological parameters such as heart rate and blood pressure, while minimizing motion-related artefacts in the sensor signal, such as a physiological signal such as heart rate.

FIG. 1C depicts an embodiment of an earpiece 101 in the form of an electrode clip. FIG. 1D depicts a clip with stimulating and reference electrodes in place on the tragus of a user. Specifically, a stimulating electrode 111 and a reference electrode 112 are embedded in a tragus clip 101 which has two lobes 101a and 101b which are biased, for example by means of a spring, to urge the lobes together so as to provide a gripping force when in place on the tragus 100 of the user of the device. In use, lobes 101a and 101b are positioned on either side of the tragus and are biased against each other to hold the tragus clip in place. The lobes 101a and 101b are positioned against the skin of the tragus. Lobe 101a includes a stimulating electrode 111 on its inner face, and the opposite lobe 101b includes a reference electrode 112 on its inner face, which are arranged to provide an electrical stimulation signal across the tragus. The lobe 101a includes a stimulating electrode, and the opposite lobe 101b includes a reference electrode, which are arranged to provide an electrical stimulation signal to the tragus. The earpiece optionally comprises a marking or a shape to indicate to a user the correct orientation of the earpiece such that the stimulating electrode is in contact with the outer surface of the tragus. For example, a marking 113 may be provided on a region of the first lobe 101a. The earpiece 101 may also include a physiological sensor 102 which is configured to record the heart rate, blood pressure, and/or temperature and store the value in a memory portion of the device. The earpiece may be configured to bring the physiological sensor 102 into contact with a region of the outer ear, such as a region of the antitragus or concha. The sensor 102 may be provided on a lobe 101a or 101b such that the sensor is held in contact with a surface of the auricle. Earpiece 101 is connected to a device that generates the electrical signal by a lead 103. The earpiece may comprise leads 103a and 103b which deliver electrical stimulation signal to the stimulating and reference electrodes, respectively.

The clip has a first gripping portion and a second gripping portion which may correspond to two lobes 101a and 101b which are biased to provide a gripping force on the tragus 100 of the user of the device. The stimulating electrode 111 is located on the first gripping portion and the reference electrode 112 is located on the second gripping portion. In some embodiments the device comprises a first and a second pair of electrodes, wherein the first pair of electrodes is configured to be placed in contact with the left tragus of the user and the second pair of electrodes is configured to be placed in contact with the right tragus of the user. Additionally, the device comprises a first earpiece and a second earpiece, and the first earpiece comprises the first pair of electrodes and the second earpiece comprises the second pair of electrodes. In some embodiments the first and the second earpieces are substantially identical and therefore the configuration shown in FIG. 1D can be applied to each ear. The device stimulation may be applied to just one ear or alternatively to both ears at the same time. The earpiece is configured to be placed in contact with an outward facing surface and an inward facing surface of the tragus. In one embodiment, the reference electrode is in contact with a first surface of the tragus and the stimulating electrode is in contact with a second surface of the tragus. In some embodiments, the stimulating electrode is a positive electrode and is in contact with a second surface of the tragus and the reference electrode is a negative electrode and is in contact with a first surface of the tragus. The first surface of the tragus may, for example, be facing inwards (i.e. towards the head of the user) and the second surface of the tragus is therefore facing outwards (i.e. away from the head of the user). In some embodiments, the first surface of the tragus may be facing outwards and the second surface of the tragus may be facing inwards.

FIG. 1E depicts a cross-section through the tragus 100 of a user with an earpiece 101 as shown in FIG. 1D in position on the tragus. The first lobe 101a is in position in contact with the outer face 100a of the tragus and the second lobe 101b is in contact with the inner face 100b of the tragus. The stimulating electrode 111 is in electrical contact with the surface of skin on the outer face of the tragus and the reference electrode 112 is in electrical contact with the surface of skin on the inner face of the tragus. Current I is shown flowing through the leads 103a and 103b connecting the electrodes 111 and 112 to the generator. A first current pathway Id is shown that is approximately direct through the tissue of the tragus and a second current pathway Is is shown that is predominantly under and close to the surface of the skin of the tragus. The device of the invention is configured such that the current resulting from the electrical stimulation signal is substantially or wholly confined to flow within the tragus, such that the current I flows at least predominantly, and in some embodiments exclusively, through the pathways Id and or Is. This is distinct from prior art devices, in which one or more electrodes are placed elsewhere on the body, such that current pathways exist that are not confined to the tragus.

The electrodes can be placed in contact with the skin via either a transcutaneous or a percutaneous contact. Where the contact is transcutaneous, this means that the electrode is placed on the skin surface but not piercing the skin. Where the contact is percutaneous this means that the electrode may have needles or electrodes that directly pierce the skin. The needle or electrode may pierce the skin to deliver the electrical stimulation signal to the user.

### Device Components and Sensors

Additional measurements can also be taken via a sensor. For example, the sensor may take sensor measurements of features such as temperature and physiological parameters. These may be used to determine the user's compliance with the device and method, for example, which may have been prescribed by a doctor or a medical member of staff. A sensor may take temperature measurements to determine whether the device is in contact with the human skin. Alternatively, the sensor could also sense the pulse of the user.

In some embodiments the device measures the physiological parameters and temperature parameters and stores these within a memory portion of the device. The measurements of these parameters are stored alongside the date and time stamp information, such that a record can be kept.

Optionally, the device may include electronic circuitry and cardiovascular function sensors to measure and monitor the voltage, current and phase relationship of the electrical stimulation signal. Measurements of current, voltage and phase relationship of the electrical stimulation signal are stored in a memory portion of the device and may be used to determine the electrical impedance of the tragus. Measurements of electrical impedance are used to sense that the electrodes are connected to a human and/or monitoring/measuring the cardiovascular function. The measurements of electrical impedance may also be stored in the memory portion of the device.

The information stored in the memory portion of the device can be used to determine the electrical stimulation signal, for example, by adjusting the form of the signal depending on the information retained in the memory. In some embodiments the memory portion of the device can be accessed remotely or by a third party.

The device may further comprise a communication module connected to the controller and the memory portion of the device. The communication module is configured to send information from the device to an external computer system and to receive information from the external computer system. This information can be used to inform the patients treatment plan and determine compliance with the prescribed treatment plan. The information received from the external computer system is used by the controller to determine the form of the electrical stimulation signal. Therefore, the electrical stimulation signal may be remotely controlled. Information shared via the communication module may include the physiological and temperature measurements taken during the use of the device. The communication module and device together form a system for modulating cerebral blood flow of a user.

The device and external computer may be further configured to act upon information from the device received by the external computer system is compared to a secondary set of information stored on the external computer system to determine a set of actions to be performed by the device and/or the external computer.

### Experimental Data

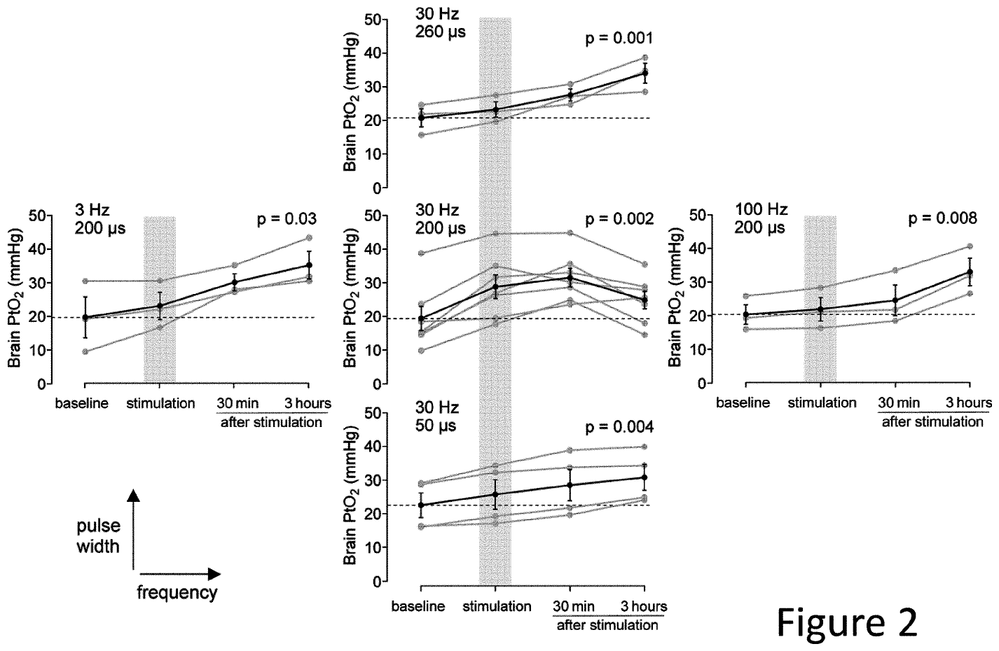

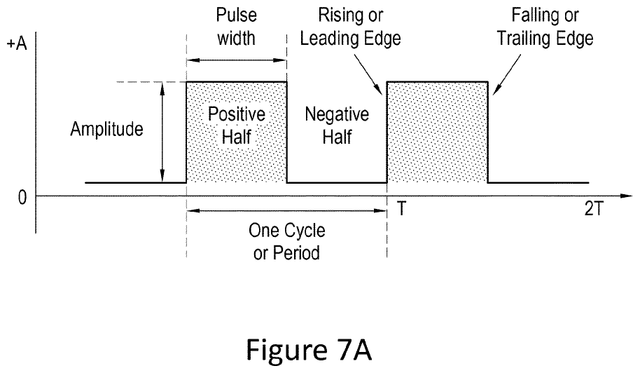

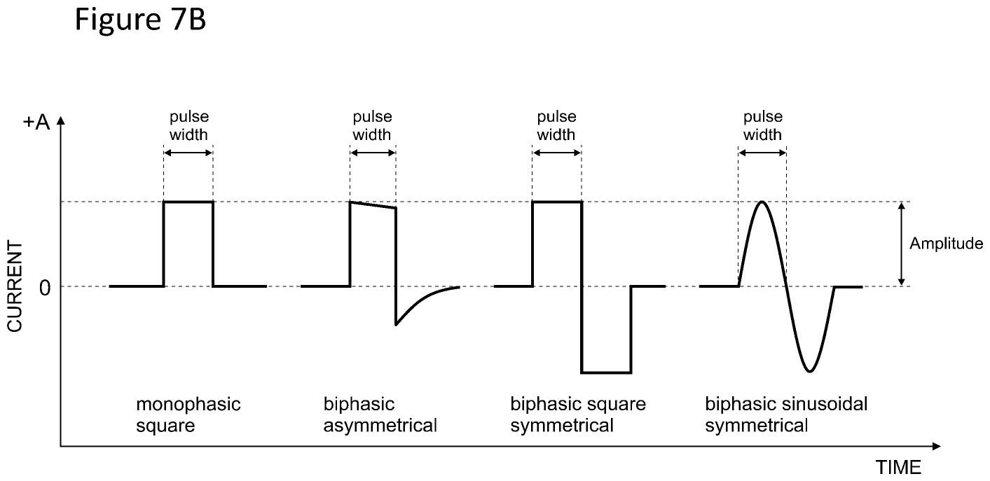

FIG. 2 depicts the values of brain tissue partial pressure of oxygen (PtO₂) recorded as a measure of cerebral blood flow, in experimental animals (laboratory rats) before, during and after transcutaneous electrical stimulation of the auricle, using the device and the method of treatment according to the invention. The study was conducted in anaesthetised (urethane, 1.3 g/kg) and artificially ventilated rats. PtO₂ was recorded in the left cerebral cortex using optical sensors based on fluorescence technology that allows real-time recordings of PtO₂. In this experimental model, changes in brain PtO₂ parallel changes in cerebral blood flow and are used as a robust measure of brain perfusion. Transcutaneous electrical stimulation of the auricle was applied for 30 minutes using the following parameters of stimulation: frequency 30 Hz, pulse width 50 microseconds; frequency 30 Hz, pulse width 200 microseconds; frequency 30 Hz, pulse width 260 microseconds; frequency 3 Hz, pulse width 200 microseconds; and frequency 100 Hz, pulse width 200 microseconds. Stimulation current was set between 1 and 3 mA. The brain PtO₂ values recorded in individual animals and means±standard errors of the mean are shown. Non-invasive neuromodulation by transcutaneous electrical stimulation of the auricle applied using a range of stimulation parameters effectively increased cerebral blood flow with a sustained effect. In this example the stimulating electrode was positioned on the skin of the outer surface of the auricle and the reference electrode was positioned on the skin of the inner surface of the auricle, and a biphasic asymmetrical pulse was used to effectively stimulate sensory nerves of the auricle (for example as shown in FIG. 7B).

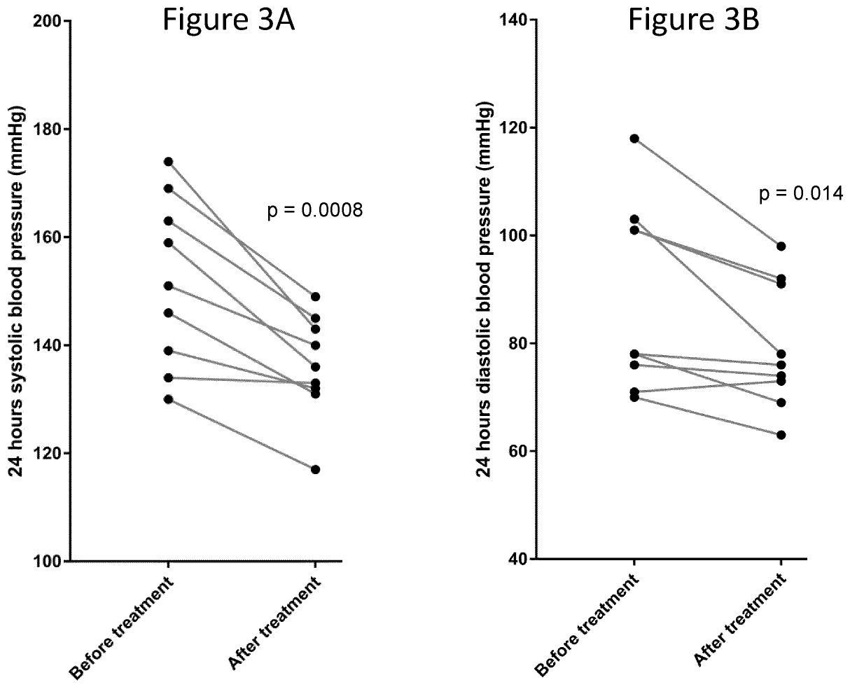

FIG. 3 depicts the blood pressure values in drug-resistant hypertensive patients (individual data, n=9) before and after the use of the device and the method according to the present invention. A study was conducted to determine the blood pressure lowering effect of transcutaneous electrical tragus stimulation, applied for up to 2 hours each day for at least 3 days in patients with drug-resistant hypertension. Drug-resistant hypertension was diagnosed in patients that displayed an office systolic blood pressure of >150 mmHg and ambulatory systolic blood pressure of ≥130 mmHg, despite adherence to maximally tolerated doses of at least three antihypertensive medications, including a diuretic. Transcutaneous electrical tragus stimulation led to a reduction of the 24 h ambulatory systolic blood pressure (p=0.0008; paired t-test; FIG. 3A) and 24 h ambulatory diastolic blood pressure (p=0.014; paired t-test; FIG. 3B) in these patients. In this example the stimulating electrode was positioned on the skin of the outer surface of the tragus and the reference electrode was positioned on the skin of the inner surface of the tragus. Bilateral stimulation using biphasic asymmetrical pulses with the following parameters was used: frequency 30 Hz, pulse width 200 microseconds, current between 1 and 8 mA.

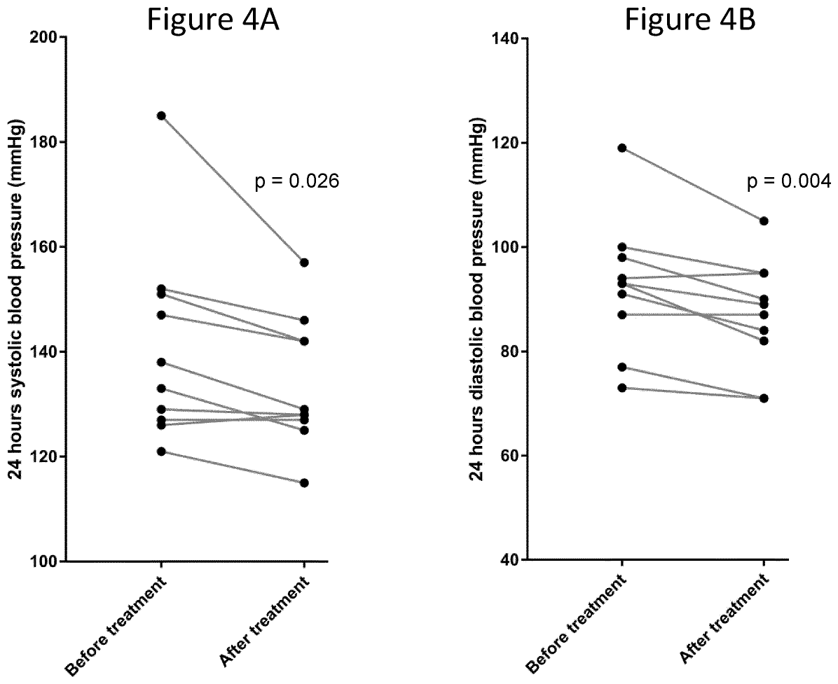

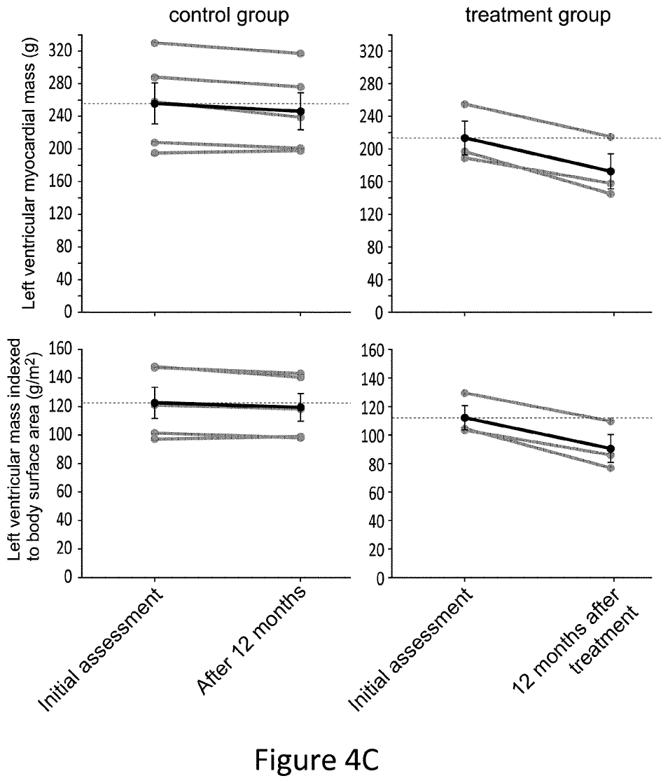

FIG. 4 depicts the blood pressure values in uncontrolled hypertensive patients (individual data, n=10) before and after the use of the non-invasive neuromodulation method according to the invention. A study was conducted to determine the blood pressure lowering effect of transcutaneous electrical tragus stimulation, applied for up to 2 hours each day for at least 3 days in patients with uncontrolled hypertension. Uncontrolled hypertension was diagnosed in patients with elevated blood pressure that were previously untreated for high blood pressure (newly diagnosed), i.e. in subjects not prescribed with any antihypertensive medications, or patients that displayed either average office systolic blood pressure ≥130 mmHg and <180 mmHg and diastolic blood pressure ≥80 mmHg (mean of two of the 3 readings), or daytime average systolic blood pressure of ≥120 mmHg and <160 mmHg and daytime average diastolic blood pressure of >80 mm Hg, despite taking up to 3 antihypertensive agents. Transcutaneous electrical tragus stimulation led to a reduction of the 24 h ambulatory systolic blood pressure (p=0.026; paired t-test; FIG. 4A) and 24 h ambulatory diastolic blood pressure (p=0.004; paired t-test; FIG. 4B) in these patients. In this example the stimulating electrode was positioned on the skin of the outer surface of the tragus and the reference electrode was positioned on the skin of the inner surface of the tragus. Bilateral stimulation using biphasic asymmetrical pulses with the following parameters was used: frequency 30 Hz, pulse width 200 microseconds, current between 1 and 8 mA.

As a specific example, clinical data demonstrate that in patients with drug-resistant hypertension (n=9) application of electrical current pulses to the skin of the tragus bilaterally, i.e. to the left and to the right tragus, for up to 2 hours each day for a minimum of 3 consecutive days resulted in a significant reduction of the 24 h ambulatory systolic blood pressure (FIG. 3A) and 24 h ambulatory diastolic blood pressure (FIG. 3B). The application of electrical current pulses to the tragus is done across the tragus, with a stimulating and a reference electrode placed on either side of the tragus. Drug-resistant hypertension was diagnosed in patients that displayed an office systolic blood pressure of >150 mmHg and ambulatory systolic blood pressure of ≥130 mmHg, despite documented adherence to maximally tolerated doses of at least three antihypertensive medications, including a diuretic. Similarly, in patients with uncontrolled hypertension (n=10) application of current pulses to the tragus bilaterally, i.e. to the left and to the right tragus, for up to 2 hours each day for a minimum of 3 consecutive days resulted in a significant reduction of the 24 h ambulatory systolic blood pressure (FIG. 4A) and 24 h ambulatory diastolic blood pressure (FIG. 4B). Uncontrolled hypertension was diagnosed in patients with elevated blood pressure that were previously untreated for high blood pressure (newly diagnosed), i.e. in subjects not prescribed with any antihypertensive medications, or patients that displayed either average office systolic blood pressure of ≥130 mmHg and <180 mmHg and diastolic blood pressure of ≥80 mmHg (mean of two of the 3 readings), or daytime average systolic blood pressure of ≥120 mmHg and <160 mmHg and daytime average diastolic blood pressure of >80 mm Hg, despite taking up to 3 antihypertensive drugs.

As another specific example, non-invasive neuromodulation in accord with the present invention was found to reduce left ventricular hypertrophy in patients with uncontrolled hypertension. FIG. 4C depicts the left ventricular mass and left ventricular myocardial mass indexed to body surface area (left ventricular mass index) in uncontrolled hypertensive patients receiving standard treatment (individual data and means±standard errors of the mean are shown, n=5) and in uncontrolled hypertensive patients (individual data and means±standard errors of the mean are shown, n=3) before and 12 months after the use of the non-invasive neuromodulation method according to the invention. A study was conducted to determine the long-term effect of transcutaneous electrical tragus stimulation on left ventricular hypertrophy (assessed by echocardiography), which is strongly associated with established hypertension. Transcutaneous electrical tragus stimulation was applied for up to 2 hours each day for 10 days. Uncontrolled hypertension was diagnosed in patients with elevated blood pressure according to the criteria described above. No differences in left ventricular mass and left ventricular mass index were observed after 12 months of observation in five patients treated in accord with the current clinical guidelines (control group of patients). Reductions in left ventricular mass and left ventricular mass index were recorded in all three patients (one man and two woman) 12 months after receiving the course of treatment using the device and the method according to the invention (treatment group of patients). In this example the stimulating electrode was positioned on the skin of the outer surface of the tragus and the reference electrode was positioned on the skin of the inner surface of the tragus. Bilateral stimulation using biphasic asymmetrical pulses with the following parameters was used: frequency 30 Hz, pulse width 200 microseconds, current between 1 and 8 mA.

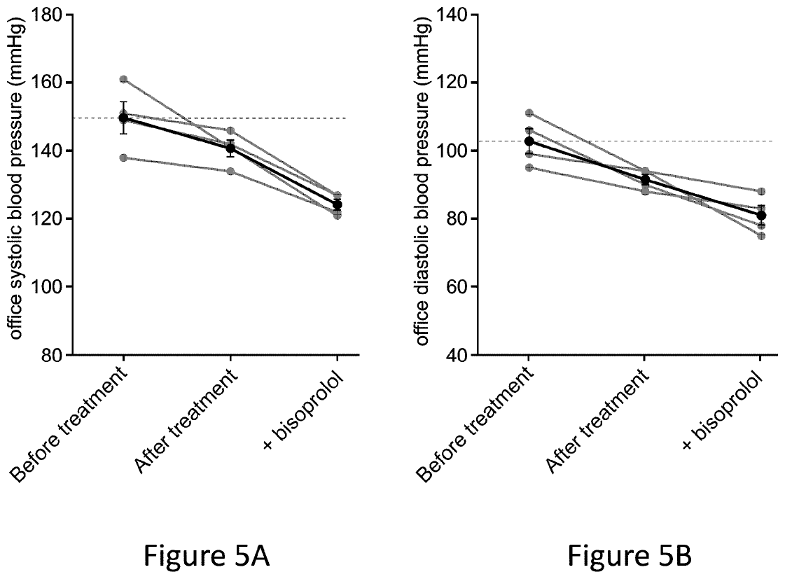

As yet another specific example, unexpectedly, non-invasive neuromodulation in accord with the present invention was found to be highly efficacious in reducing arterial blood pressure when applied in combination with pharmacological treatment involving beta-adrenoceptor blockade, even when the dose of a β-blocker used was much lower than the effective therapeutic dose required to lower blood pressure when a β-blocker is given on its own (Cochrane Database Syst Rev. 2016 3: CD007451). FIG. 5 illustrates the blood pressure values in uncontrolled hypertensive patients (individual data and means±standard errors of the mean are shown, n=4) before and after the use of the device and the method of treatment according to the invention in combination with pharmacological treatment using a beta-adrenoceptor antagonist bisoprolol. A study was conducted to determine the blood pressure lowering effect of non-invasive neuromodulation according to the invention applied for up to 2 hours each day for at least 3 days in combination with bisoprolol (3 patients received 1.25 mg per day; 1 patient received 5 mg per day) in patients with uncontrolled hypertension. Uncontrolled hypertension was diagnosed in patients with elevated blood pressure according to the criteria described above. Electrical stimulation of the left and the right tragi applied for up to 2 hours each day for a minimum of 3 consecutive days led to a reduction of the office systolic blood pressure (by 9 mmHg; p=0.047; paired t-test; FIG. 5A) and office diastolic blood pressure (by 11 mmHg; p=0.017; paired t-test; FIG. 5B). In this example the stimulating electrode was positioned on the skin of the outer surface of the tragus and the reference electrode was positioned on the skin of the inner surface of the tragus. Bilateral stimulation using biphasic asymmetrical pulses with the following parameters was used: frequency 30 Hz, pulse width 200 microseconds, current between 1 and 8 mA. Non-invasive neuromodulation in accord with the invention was found to have a therapeutic effect in lowering systemic blood pressure in combination with use of a beta-adrenoceptor antagonist. The effect of electrical stimulation of the tragus was greatly potentiated in patients following treatment with the beta-adrenoceptor antagonist bisoprolol, leading to a further reduction of the office systolic blood pressure (by a further 16 mmHg; p=0.001; paired t-test; FIG. 5A) and office diastolic blood pressure (by a further 10 mmHg; p=0.023; paired t-test; FIG. 5B). Thus, combination of non-invasive neuromodulation by bilateral transcutaneous electrical tragus stimulation with systemic beta-adrenoceptor blockade reduced systolic and diastolic blood pressure in previously uncontrolled hypertensive patients by 25 mmHg and 22 mmHg, respectively. The dose of bisoprolol used in this trial is below the level that is expected to have any therapeutic effect in reducing blood pressure (Cochrane Database Syst Rev. 2016 3: CD007451) when used alone. Therefore, the effect of the combination of non-invasive neuromodulation by bilateral transcutaneous electrical tragus stimulation with systemic beta-adrenoceptor blockade in reducing systemic blood pressure is greater than the sum of the effects of each treatment when applied separately.

In patients with paroxysmal AF application of electrical current pulses to the skin of the tragus bilaterally reduced the frequency and duration of AF episodes.

To achieve a therapeutic effect manifested as a sustained reduction of arterial blood pressure in hypertensive patients, improved cardiac function in heart failure, or reduction in AF burden, electrical stimulation of the nerves innervating the tragus required application of current pulses with the following specific parameters: frequency 1-30 Hz, amplitude 0.1-8 mA, pulse width 10-250 microseconds, square shape monophasic or biphasic asymmetrical or biphasic symmetrical pulse. Transcutaneous application of electrical current pulses at frequencies between 1-30 Hz, amplitudes between 0.1-8 mA, and pulse widths between 10-250 microseconds triggers reliable action potential firing in the subcutaneous nerve fibers innervating the tragus, resulting in neuromodulation and improvement of cerebral blood flow, as illustrated by FIG. 2. Application of current pulses at frequencies lower or higher than the 1-100 Hz range, amplitudes smaller or higher than 0.1-8 mA range, and pulse widths shorter or longer 10-500 microseconds range is without the therapeutic effect. Moreover, to achieve a sustained reduction of arterial blood pressure in hypertensive patients, neuromodulation by electrical stimulation of the tragus requires a course of treatment involving several sessions of stimulation in accord with the following stimulation treatment programme: stimulation is applied daily to the left and right tragi simultaneously (i.e. bilaterally) for a period of between 5 min and 2 hours each day for a minimum of 3 consecutive days (initial course of treatment). Then the stimulation may be applied once a week (every 7 days) to the left and right tragi simultaneously for a period of up to 2 hours each session during the course of treatment (subsequent course of treatment). The stimulation can alternatively be applied several times a day. For example, the device can be applied to the user throughout several time periods in the day. The total sum of all daily usage of the device may add up to a period of between 5 min and 2 hours each day.

The reduction in arterial blood pressure following the initial course of treatment has been found to persist for several weeks with or without the patient receiving the subsequent course to treatment. If after the initial course of treatment, the blood pressure remains elevated, further initial courses of treatment, followed by (or not as required by a patient), subsequent courses of treatment may be administered to achieve and maintain the therapeutic effect. This cycle of treatments may be applied on a regular basis for as long as the hypertension condition exists, and/or the therapeutic benefit supports the well-being of a patient.

### Patient Screening

It is possible to screen patients to identify those patients who are potential responders to treatment in accord with the present invention. To achieve this a patient's electrocardiogram is recorded for a minimum period of 1 min and the power spectrum of heart rate variability is analysed to determine the low frequency (LF) to high frequency (HF) ratio (LF/HF) of heart rate variability spectrum. The method of neuromodulation via stimulation of the sensory innervation of the tragus is expected to reduce blood pressure and left ventricular hypertrophy in hypertensive patients and improve cardiac function in patients with heart failure whose LF/HF ratio of heart rate variability spectrum is larger than 1 (one).

It is also possible to screen patients to identify those patients who are potential responders to treatment in accord with the present invention by the assessment of their heart rate recovery after standing. To perform this test, the patients are asked to rest comfortably in the supine position for 10 minutes before performing a stand-up test. The supine position means lying horizontally with the face and torso facing up. The supine baseline values of heart rate, systolic and diastolic blood pressure are recorded. Patients are asked to stand up in a timely manner (<5 s). Heart rate, systolic and diastolic blood pressure recordings are taken at 10 seconds time intervals for about 1 minute after the stand. Differences from the baseline measures are calculated by subtracting baseline resting heart rate values (supine baseline values) from heart rate values obtained at each time point during standing. The method of neuromodulation via stimulation of the sensory innervation of the tragus is expected to reduce blood pressure in hypertensive patients and improve cardiac function in patients with heart failure whose heart rate recovery between 10 s and 20 s after standing (measured as the difference from the peak heart rate value immediately after standing) is less than 6 (six) beats per minute.

### Device Components

The device for modulating cerebral blood flow to treat diseases of the circulatory system includes at least one, and in an example two earpieces, each comprising a pair of electrode(s) configured to provide an electrical stimulation signal to the tragus, such as a stimulating electrode and a reference electrode configured such that the electrical current flows across the tragus, a generator connected to the pairs of electrodes for generating the electrical stimulation signal, a controller connected to the generator, for determining both the form of the electrical stimulation signal and the pattern of stimulation. Optionally, the device may include electronic circuitry and cardiovascular function sensors to measure and monitor the voltage and current of the applied electrical signal as well as cardiovascular physiological signals (e.g. ECG, blood pressure); a micro-controller or computer, memory, user input keypads and peripheral devices, physiological sensors, display element, and associated circuitry to input, control and record data associated with the use of the device, a wireless and/or wired communications system for interfacing with external devices, and a computer, tablet and/or smartphone external to the device to enable the device to be programmed, and/or transfer of data and information to and from the device. In this way, a patient's cerebral blood flow is expected to be improved, arterial blood pressure and left ventricular hypertrophy is expected to be reduced and cardiac function improved by using the device and receiving the course of treatment involving the stimulation of sensory innervation of the tragus in accord with the present invention. In an example embodiment, the device comprises a first and a second earpiece, and the stimulation of sensory innervation of the tragus is performed bilaterally.

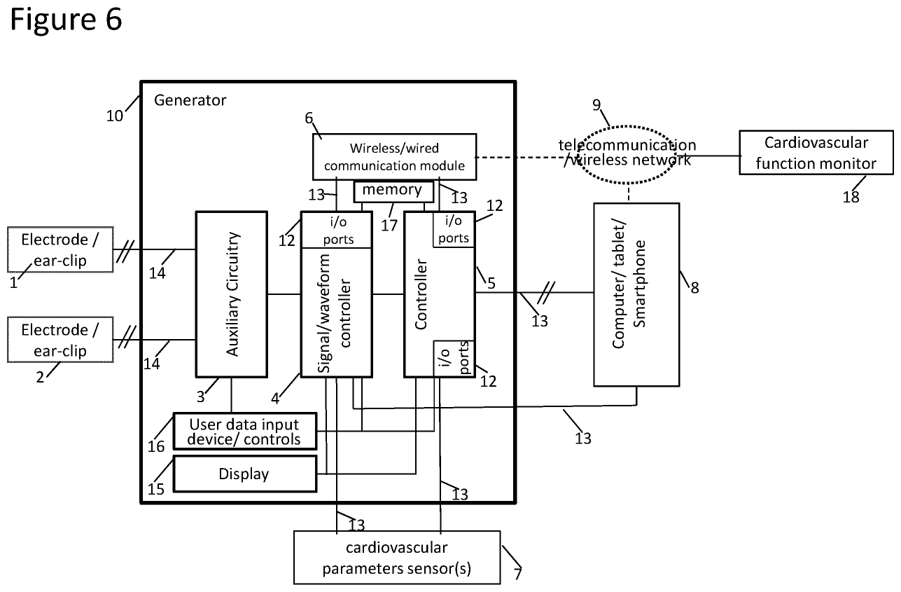

FIG. 6 depicts a block diagram for a device according to the present invention. The generator 10 may comprise a signal/waveform generator 4, a controller 5, memory 17, auxiliary circuitry 3, user data input/control device(s) 16 (such as keypads, dials, actuator/switches), display device 15, communication modules (wireless and/or wired communication), 6. The illustrated generator 10 further includes a transceiver or communication module and other input/output circuit(s) (i/o ports) 12. The i/o ports allow the generator device to communicate with other devices 8, and thus can be used to program the generator device and/or upload historical generator data recorded over a period of time, for example. The i/o ports 12 may include a switch (such as mechanical, electrical, electronic and magnetic) providing a means for initiating a programmed stimulation algorithm which may be triggered by a physician, healthcare professional or patient.

The generator 10 delivers an electrical stimulation signal (determined by the stimulation algorithm) using a defined schedule to modulate cerebral blood flow and lower arterial blood pressure, reduce left ventricular hypertrophy, and/or reduce AF burden and improve cardiac function of the user. According to various embodiments, the device further includes at least one port 12 which may be part of the controller 4 and/or the micro-controller 5 to connect to at least one lead 13 (FIG. 6). Thus, for example, the lead(s) 13 is/are capable of detaching from the device 10, and other leads are capable of being used with the device. The lead 13 may be used to connect to physiological and, or temperature sensors. As is described above, the generator is for determination of the electrical stimulation signal. More specifically, the generator may be for the determination of time-course parameters related to the stimulation algorithm, such as pulse width, pulse frequency, waveform and waveform pattern herein referred to as the waveform. Examples of the waveform pattern include, but not restricted to, sinusoidal, square, triangular, biphasic symmetrical, biphasic asymmetrical and “white noise” signals. FIG. 7B illustrates some examples of stimulation waveform that can be used to transcutaneously stimulate the sensory innervation of the tragus. The controller in an example produces the electrical parameters of the stimulation algorithm (current and/or voltage amplitude, frequency, burst-frequency, waveform and duration) of the stimulation algorithm, herein referred to as the waveform parameters, based on the signal determined by and received from the generator. The generator may also determine the waveform parameters based on a signal received from a sensor. This determination may take place in three ways: user-controlled, utilising the display 15 and/or user data input/control device(s) 16, or automatically from pre-programmed, computer-readable instructions determined by a micro-controller, or programmed, computer-readable instructions determined by an external computer 8.

The controller 4, micro-controller 5, memory 17, auxiliary circuitry 3, user data input/control device(s) 16 (such as keypads, dials switches), display device 15, communication modules (wireless and/or wired communication), 6 may be located within the same component, which may be a portable battery operated electronic device. The portable electronic device is for example able to run applications or apps, and is for example a laptop computer, a tablet or a smartphone 8. Alternatively, the generator may be in the form of a portable electronic device, and the generator may be a separate component.

The controller 4 may further be connected to the Auxiliary Circuit 3 to provide an electrical stimulation signal to at least one pair of electrodes 1 & 2 to stimulate at least one tragus and in an example embodiment, both tragi of a human subject ear(s) when an appropriate signal is provided to the electrode or electrodes.

A stimulation algorithm is provided using a single lead and a single electrode on the lead. However, multiple leads and multiple electrodes on the leads can be used. The electrode(s) and/or 1 & 2 may be of a wearable device is for example connected to the generator device 10 via an electric cable 14, or via a wireless connection 14 such as Bluetooth. Some embodiments where more than one electrode is used to stimulate the patient, the same or different waveforms may be applied to two or more electrodes. The two different waveforms may vary in pattern and/or waveform parameters.

The generator may be an open loop or closed loop system and controlled by computer-readable instructions. In the closed loop embodiment the stimulation algorithm may be adapted in response to signals from the cardiovascular parameters sensor(s) 7 which may include one or a plurality of sensors such as a blood pressure sensor, temperature sensor, pulse oximeter, electrocardiogram sensor, heart rate sensor, temperature and tissue impedance sensor designed to sense a parameter indicative that the electrodes are connected to a human, and/or monitoring/measuring the cardiovascular function where the stimulation algorithm is adapted to chronically lower blood pressure using the sensed parameter. Thus, the closed loop system is capable of employing information from the cardiovascular sensors as a feedback mechanism and/or programme to reduce and/or increase the stimulation intensity, alter and/or change the waveform and/or waveform parameters as appropriate to maintaining some measured physiological parameters within an upper and lower boundary during the stimulation. In the open loop embodiment, the stimulation algorithm is adapted to chronically lower blood pressure and/or adjusting the waveform parameters using an external device 8. Additionally, in various embodiments, the generator is adapted to set parameters of the stimulation signal and, in some embodiments, vary parameters of the stimulation algorithm to adjust the intensity of the stimulation using either the user data input device 16 and/or an external computer 8.

The memory 11 (or memory portion) includes computer-readable instructions that are capable of being operated on by the controller and/or micro-controller to perform functions of the device. Thus, in various embodiments, the generator is adapted to operate on the instructions to provide an electrical stimulation signal based on a programmed stimulation algorithm to deliver a therapy such as anti-hypertensive, heart failure, left ventricular hypertrophy and atrial fibrillation improvement therapies. Additionally, in various embodiments, the generator is adapted to set parameters of the stimulation signal and, in some embodiments, vary parameters of the stimulation signal to adjust the intensity of the electrical stimulation signal, such as is generally illustrated by the stimulation intensity as illustrated in FIG. 7A.

The micro-controller and memory devices may include pre-programmed computer-readable instructions to provide controlled electronic access to the generator, implement security, password and encryption feature to limit access to the device and stored data and store information such as the user and maintenance instruction, device specific data required by legal and/or regulatory statutes.

According to various embodiments, a single or plurality of physiological parameters of a patient may be measured by means of sensors, such as cardiovascular sensors, and recorded by the device. Further, several such measurements may be made at different times during a single stimulation period or at different times over more than one stimulation period to establish the value and/or range of values for any particular physiological parameters. In one such embodiment, the magnitude of the voltage and/or current of the electrical stimulation signal and their phase relationship may be used to determine the electrical impedance of the patient's skin and be recorded by the device. Thus, value or range of values of such physiological parameters recorded during the stimulation algorithm period in conjunction with the usage of the device (date, time, waveform and waveform parameters) may be recorded and combined to construct a data-set (individual usage data), indicative of the patient's usage of the device, thus providing and recording information on the use of the device by a patient. This information may further be reported to the external device such that the information may be used to monitor a patient's condition, or to demonstrate and/or provide evidence of a patient's compliance with the stimulation/treatment programme as prescribed by a physician, healthcare professional or healthcare agreement, contract or health insurance agreement with a third party. Further, in some embodiments the individual usage dataset may be accessed remotely by means of the communication module to enable remote monitoring of patients, validating a patient's compliance with the healthcare plan and prescribed treatment, adapting initial and subsequent course of stimulation and the stimulation algorithm to change the prescribed treatment.

According to various embodiments, the device may communicate with an external computer, tablet or smartphone whereby by the computer, tablet or smartphone is further able to communicate with a cardiovascular function monitor such as a blood pressure monitor and/or heart-rate monitor and/or ECG monitor to record individual usage data and data from the cardiovascular function monitor(s). A physician, or a healthcare professional may examine a patient's cardiovascular variables, advise the patient to, or remotely (via the internet or other telecommunication/computer network) on the initial course of treatment and/or subsequent course of treatment and/or determine a set of actions to be performed by the device and/or the external computer.

According to various embodiments, the individual usage data may be analysed to determine if a patient had used the device as prescribed by a physician or a healthcare professional and/or complied with the terms of any healthcare or medical insurance policy agreement(s) and thus used to determine whether any financial penalties, changes in insurance premium or benefits financial or otherwise may be paid or accrue to individual.

The device may include a plurality of stimulation electrodes on one earpiece and may include a reference electrode associated with each stimulation electrode, or a single reference electrode associated with the plurality of stimulation electrodes on a given earpiece. Specifically, there may be a plurality of stimulating electrodes configured to provide an electrical stimulation signal to the tragus.

### Pulse Waveform

As depicted on FIG. 7A, the electrical stimulation signal has an amplitude, waveform, a pulse width and a frequency. The amplitude is the magnitude or intensity of the signal waveform measured in volts or amps (measured by the difference between the highest and lowest part of the waveform). The frequency is the number of times the waveform repeats itself within a one second time period, measured in Hz. The pulse width is the length of time in seconds that the waveform takes to repeat itself from start to finish. A square wave is illustrated in FIG. 7A. FIG. 7B illustrates some examples of stimulation pulse waveform that can be used to transcutaneously stimulate the sensory innervation of the tragus, however, in accord with the present invention the waveform can take any shape including a sinusoidal, square, triangular, biphasic or ‘white noise’ waveform.

The generated waveform can be a symmetrical monophasic waveform, or a symmetrical biphasic waveform, or a symmetrical triphasic waveform. The generated waveform can be an asymmetrical monophasic waveform, or an asymmetrical biphasic waveform, or an asymmetrical triphasic waveform. It is feasible for the waveform to take any of these shapes.

In some embodiments the electrical stimulation signal comprises a cyclically repeating multiphasic pulse waveform in which the amplitude and/or duration of the phases of the pulse at which the signal at the stimulating electrode is positive with respect to the signal at the reference electrode is/are greater than the amplitude and/or duration of the phases of the pulse at which the signal at the stimulating electrode is negative with respect to the signal at the reference electrode.

In this way, a net conventional current flow is provided over the duration of the pulse that is positive from the stimulating electrode to the reference electrode.

In some embodiments the electrical stimulation signal comprises a cyclically repeating multiphasic pulse waveform in which the amplitude and/or duration of the phases of the pulse at which the signal at the stimulating electrode is positive with respect to the signal at the reference electrode is/are less than the amplitude and/or duration of the phases of the pulse at which the signal at the stimulating electrode is negative with respect to the signal at the reference electrode.

In this way, a net conventional current flow is provided over the duration of the pulse that is negative from the stimulating electrode to the reference electrode.

In some embodiments the multiphasic pulse waveform is a biphasic pulse waveform. In some embodiments the multiphasic pulse waveform is a triphasic pulse waveform.

In some embodiments the electrical stimulation signal comprises a cyclically repeating pulse waveform comprising a plurality of pulses, in which the amplitude and/or duration of the pulses during which the signal at the stimulating electrode is positive with respect to the signal at the reference electrode is/are greater than the amplitude and/or duration of the pulses during which the signal at the stimulating electrode is negative with respect to the signal at the reference electrode.

In this way, a net conventional current flow is provided over the duration of the plurality of pulses that is positive from the stimulating electrode to the reference electrode.

In some embodiments the electrical stimulation signal comprises a cyclically repeating pulse waveform comprising a plurality of pulses, in which the amplitude and/or duration of the pulses during which the signal at the stimulating electrode is positive with respect to the signal at the reference electrode is/are less than the amplitude and/or duration of the pulses during which the signal at the stimulating electrode is negative with respect to the signal at the reference electrode.

In this way, a net conventional current flow is provided over the duration of the plurality of pulses that is negative from the stimulating electrode to the reference electrode.

### Methods

There is provided a method for non-invasive electrical stimulation of nerves that project to the skin of the outer ear using a device as disclosed herein, comprising the steps of: bringing the stimulating electrode and the reference electrode into contact with the tragus of a user; producing, using the device, an electrical stimulation signal applied to the stimulating electrode and the reference electrode; and determining, using the controller, the waveform and the frequency of the electrical stimulation signal, wherein: the electrical stimulation signal comprises a series of electrical pulses, each pulse repeating with a frequency of about 1 Hz to about 100 Hz and each pulse has a duration of about 10 microseconds to about 500 microseconds and an amplitude of about 0.1 mA to about 20 mA.

In some embodiments the parameters of the frequency, the pulse duration and the amplitude of a pulse are each selected in a range as disclosed herein.

In some embodiments, the method comprises applying the electrical stimulation signal to the tragus of the user such that the current flow between the stimulating electrode and the reference electrode is primarily through the tissue of the tragus, and negligibly through tissue that does not form part of the tragus.

In some embodiments the method comprises applying the electrical stimulation signal to the tragus of the user such that the current flow between the stimulating electrode and the reference electrode is primarily or exclusively between the outer and the inner surfaces of the tragus, through the tissue of the tragus.

In some embodiments the electrical stimulation signal comprises a cyclically repeating series of pulses; and the electrical stimulation signal is selected such that during each cycle there is a net conventional current flow from the stimulating electrode to the reference electrode.

In some embodiments the electrical stimulation signal is selected such that over the course of a series of cyclically repeating pulses, there is a net conventional current flow from the stimulating electrode to the reference electrode.

In some embodiments the net conventional current flow is positive. In other embodiments the net conventional current flow is negative.

In some embodiments the electrical stimulation signal comprises a cyclically repeating multiphasic pulse waveform in which the amplitude and/or duration of the phases of the pulse at which the signal at the stimulating electrode is positive with respect to the signal at the reference electrode is/are greater than the amplitude and/or duration of the phases of the pulse at which the signal at the stimulating electrode is negative with respect to the signal at the reference electrode. In this way, a net conventional current flow is provided over the duration of the pulse that is positive from the stimulating electrode to the reference electrode.

In some embodiments the electrical stimulation signal comprises a cyclically repeating multiphasic pulse waveform in which the amplitude and/or duration of the phases of the pulse at which the signal at the stimulating electrode is positive with respect to the signal at the reference electrode is/are less than the amplitude and/or duration of the phases of the pulse at which the signal at the stimulating electrode is negative with respect to the signal at the reference electrode.

In this way, a net conventional current flow is provided over the duration of the pulse that is negative from the stimulating electrode to the reference electrode.

In some embodiments the multiphasic pulse waveform is a biphasic pulse waveform. In some embodiments the multiphasic pulse waveform is a triphasic pulse waveform.

In some embodiments the electrical stimulation signal comprises a cyclically repeating pulse waveform comprising a plurality of pulses, in which the amplitude and/or duration of the pulses during which the signal at the stimulating electrode is positive with respect to the signal at the reference electrode is/are greater than the amplitude and/or duration of the pulses during which the signal at the stimulating electrode is negative with respect to the signal at the reference electrode. In this way, a net conventional current flow is provided over the duration of the plurality of pulses that is positive from the stimulating electrode to the reference electrode.

In some embodiments the electrical stimulation signal comprises a cyclically repeating pulse waveform comprising a plurality of pulses, in which the amplitude and/or duration of the pulses during which the signal at the stimulating electrode is positive with respect to the signal at the reference electrode is/are less than the amplitude and/or duration of the pulses during which the signal at the stimulating electrode is negative with respect to the signal at the reference electrode.

In this way, a net conventional current flow is provided over the duration of the plurality of pulses that is negative from the stimulating electrode to the reference electrode.

### Optimised (Example) Treatment Programmes

To achieve a therapeutic effect manifested as a sustained reduction of blood pressure in hypertensive patients, or reduction of AF burden, or improved cardiac function in heart failure, electrical stimulation of the sensory innervation of the tragus has been found to be desirable to be applied using the following specific parameters and ranges: frequency 1-30 Hz, amplitude 0.1-8 mA, pulse width 10-250 microseconds. It is desirable to apply stimulation with a square shaped monophasic or biphasic symmetrical or asymmetrical pulses bilaterally, i.e. to the left and right tragi simultaneously. Based on the results from the experiment illustrated by FIG. 2, it is believed that the therapeutic effect is associated with an improvement in cerebral blood flow and may result from such improvement. In particular, the therapeutic effect can be achieved by using a frequency of no less than 1 Hz and no more than 100 Hz. Furthermore, the therapeutic effect can also be observed by using a frequency of no less than 3 Hz and no more than 50 Hz. The therapeutic effect can be achieved by using a pulse width of no less than 10 microseconds, and no more than 500 microseconds and amplitude of no less than 0.1 mA, and no more than 8 mA. The therapeutic effect can also be achieved by using a pulse width of no less than 100 microseconds, and no more than 500 microseconds In some embodiments of the method, it is possible to optimise just one of the parameters of stimulation, such as frequency, amplitude or pulse width, and it is not required to optimise all to produce a therapeutic effect. For example, according to the embodiment the electrical stimulation signal may have a pulse that repeats with a frequency of 1 Hz to 100 Hz or each pulse may have a duration of 10 microseconds to 500 microseconds or may have an amplitude of 0.1 mA to 20 mA.

For completeness we submit that in some embodiments, the electrical stimulation signal comprises a series of electrical pulses, each pulse repeating with a frequency in the range about 3 Hz to about 50 Hz and each pulse having a duration of about 100 microseconds to about 500 microseconds and an amplitude of about 0.1 mA to about 8 mA.

In some embodiments the frequency is in the range from about 1 Hz to about 100 Hz, such as about 1 Hz to 10 Hz, 10 Hz to 20 Hz, 20 Hz to 30 Hz, 30 Hz to 40 Hz, 40 Hz to 50 Hz, 50 Hz to 60 Hz, 60 Hz to 70 Hz, 70 Hz to 80 Hz, 80 Hz to 90 Hz, or 90 Hz to about 100 Hz.

In some embodiments the frequency is in the range 3 Hz to 20 Hz, 5 Hz to 30 Hz, 10 Hz to 50 Hz, 15 Hz to 60 Hz, 20 Hz to 75 Hz, 25 Hz to 80 Hz, 30 Hz to 100 Hz.

In some embodiments the frequency is in the range 3 Hz to 50 Hz. In some embodiments the frequency is in the range about 3 Hz to about 35 Hz.

In some embodiments the pulse has a duration in the range about 10 microseconds to about 500 microseconds, such as about 10 microseconds to 100 microseconds, 20 microseconds to 200 microseconds, 30 microseconds to 300 microseconds, 40 microseconds to 400 microseconds, 50 microseconds to about 500 microseconds.

In some embodiments the pulse has a duration in the range 100 microseconds to 200 microseconds, 200 microseconds to 300 microseconds, 300 microseconds to 400 microseconds, 400 microseconds to 500 microseconds.

In some embodiments the pulse has a duration in the range 50 microseconds to 200 microseconds, 100 microseconds to 250 microseconds, 200 microseconds to 500 microseconds.

In some embodiments the pulse has a duration in the range 100 microseconds to 500 microseconds. In some embodiments the pulse has a duration in the range about 100 microseconds to about 300 microseconds.

In some embodiments the amplitude is in the range about 0.1 mA to about 10 mA, such as about 0.1 mA to about 2 mA, about 0.2 mA to about 5 mA or about 0.5 mA to about 10 mA.

In some embodiments the amplitude is in the range 0.1 mA to 1 mA, 0.2 mA to 2 mA, 0.3 mA to 3 mA, 0.4 mA to 4 mA, 0.5 mA to 5 mA, 0.6 mA to 6 mA, 0.7 mA to 7 mA, 0.8 mA to 8 mA, 0.9 mA to 9 mA or 1.0 mA to 10 mA.

In some embodiments the amplitude is in the range 0.1 mA to 5 mA, 0.5 mA to 8 mA or 1 mA to 10 mA.

In some embodiments the amplitude is in the range about 0.5 mA to about 5 mA.

In some embodiments the amplitude is in the range about 0.1 mA to about 20 mA.

To modulate cerebral blood flow in order to achieve a reduction of blood pressure in hypertensive patients, and/or left ventricular hypertrophy, neuromodulation by electrical stimulation of the sensory innervation of the tragus requires a course of treatment involving several sessions of stimulation in accord with the following stimulation treatment programme: stimulation is applied daily to the left and right tragi simultaneously (i.e. bilaterally) for a period of between 5 min and 2 hours each day for a minimum of 3 consecutive days (initial course of treatment). The therapeutic effect can be optimised by applying the method of electrical tragus stimulation to the user for a minimum of 5 minutes and a maximum of 2 hours per day. The electrical stimulation is applied to the user separated by intervals of at least one day. Then the stimulation may be applied once a week (every 7 days) to the left and right tragi simultaneously for a period of up to 2 hours each session during the course of treatment (subsequent course of treatment).

Additionally, further treatment plans shown to be effective involve applying the electrical stimulation of the sensory innervation of the tragus of the user using different periods. During a first period, the method is applied to the user for between 5 minutes and 2 hours each day. The first period is typically a minimum of 3 consecutive days, although stimulations can be applied for more consecutive days depending on the needs of the patient to achieve a therapeutic effect. During a second period the method is stopped for at least 2 days. During a third period the method is applied to the user for between 5 minutes and 2 hours each day.

The use of the device and the method of treatment in accord with the present invention may be applied to the user in combination with any medications administered according to the clinical guidelines for modulation of the pharmacological effect.

Furthermore, in an embodiment of the method the treatment programme of the user is adjusted in response to measurements of their blood pressure. The treatment can involve continuous measurement of the patient's blood pressure followed by comparison to a predetermined threshold value (for example, the level of blood pressure considered to be healthy). The threshold value may be set by the user or by a third-party controller. The third-party controller may communicate with the device via the communication module. Firstly, measurements of the user's blood pressure are taken and recorded in the memory portion of the device. Then the controller determines whether the user's blood pressure is greater than a predetermined threshold value; and if the user's blood pressure is greater than the predetermined threshold value, the generator is instructed to produce the electrical stimulation signal.

Currently the only regulatory approved medical treatment for hypertension includes the consumption of pharmaceutical agents, which do not work for some patients. The other big challenge is that hypertension is a life-long condition and requires patients to take daily medication for the rest of their life. Many patients (45% of all medicated patients) do not take their medications as prescribed, in part due to side-effects or poor adherence. The technical benefit of the claimed device-based treatment solution is that it works for drug-resistant patients and patients that are uncontrolled on medications. Also, the claimed solution means that it is possible to treat patients for a short period or implant the device and it continues to work without the patient having to do anything for a long time or having to remember to take a regular prescription. In the present case it has been found that the treatment involving stimulation of the sensory innervation of the tragus can be applied to a patient for between 3 days to 2 weeks and their blood pressure remains reduced for several weeks after the initial course of treatment with some patients maintaining reduced blood pressure for up to 12 months after the initial course of treatment. After this the treatment can be repeated. The claimed treatment solution can be used in combination with all prescribed pharmaceutical agents.

Another benefit of the device-based treatment solution is that it is possible to check that the device has been used so the health care practitioner can monitor whether a patient has used it and complied with their prescribed treatment. This monitoring can be carried out remotely. The only way one can do this with drugs is through a blood/urine testing which is time-consuming and costly. Compliance to medical treatment is a big issue for health insurance and public health funders, as maintaining blood pressure within the recommended range significantly reduces a patient's risk of stroke, myocardial infarction, kidney failure and dementia, thus reducing significant life-long associated health and social care costs.

Features of the above aspects can be combined in any suitable manner. It will be understood that the above description is of specific embodiments by way of aspect only and that many modifications and alterations will be within the skilled person's reach and are intended to be covered by the scope of the appendant claims.

1. A device for modulating cerebral blood flow of a user, the device comprising:
a generator configured to produce an electrical stimulation signal;
a controller, connected to the generator, configured to determine the form of the electrical stimulation signal;
an earpiece, connected to the generator and controller, the earpiece comprising an electrode, wherein the controller transmits the electrical stimulation signal to the electrode;
wherein the electrode is configured to be placed in contact with and provide the electrical stimulation signal to the nerves innervating a tragus of the user,
wherein the electrical stimulation signal comprises a series of electrical pulses, each pulse repeats with a frequency of 1 Hz to 100 Hz and each pulse has duration of 10 microseconds to 500 microseconds and an amplitude of 0.1 mA to 8 mA,
wherein the device is used by the user at least once a day for 3 consecutive days.

2. The device of claim 1, wherein modulating cerebral blood flow is applied to the user for any of the following purposes: reducing systemic arterial blood pressure, reducing left ventricular hypertrophy, reducing pulmonary arterial blood pressure, treating heart failure and/or treating atrial fibrillation.

3. The device of any preceding claim, wherein the electrical stimulation signal is applied transcutaneously to the user.

4. The device of claim 1 or 2, wherein the electrical stimulation signal is applied percutaneously to the user.

5. The device of any preceding claim, wherein the device is applied to the user for a minimum of 5 minutes and a maximum of 2 hours per day.

6. The device of any preceding claim, wherein the device comprises at least a first and a second electrode, wherein the first electrode is configured to be placed in contact with the left tragus of the user and the second electrode is configured to be placed in contact with the right tragus of the user.

7. The device of claim 6, wherein the device comprises a first earpiece and a second earpiece and the first earpiece comprises the first electrode and a first reference electrode, and the second earpiece comprises the second electrode and a second reference electrode.

8. The device of any preceding claim further including a securing means configured to secure the electrode to a tragus of a user.

9. The device of claim 8 wherein the securing means includes a clip.

10. The device of claim 9 wherein the clip has a first gripping portion and a second gripping portion which are biased into contact with each other.

11. The device of claim 10, wherein the electrode is located on the first gripping portion.

12. The device of claim 10 or 11, wherein a reference electrode is located on the second gripping portion.

13. The device of any preceding claim, further including a physiological sensor configured to measure the value of a physiological parameter and store the value in a memory portion of the device.

14. The device of any preceding claim, further including a temperature sensor configured to measure the temperature of the skin of the tragus and store the value in a memory portion of the device.

15. The device of claim 13 or 14, wherein the value stored in the memory portion is used by the controller to determine the form of the electrical stimulation signal.

16. The device of any of claims 13 to 15, wherein the physiological sensor and/or the temperature sensor is located on the clip.

17. The device of any preceding claim, wherein physiological sensor measurements, temperature sensor measurements and time and date information on the use of the device by the patient is recorded and stored in a memory portion of the device.

18. The device of any preceding claim, wherein measurements of current, voltage and phase relationship of the electrical stimulation signal are stored in a memory portion of the device and are used to determine the electrical impedance of the tragus.

19. The device of claim 18, wherein electrical impedance of the tragus and time and date information on the use of the device by the patient are recorded and stored in a memory portion of the device.

20. The device of any preceding claim, wherein the controller is configured to produce the electrical stimulation signal and the pattern of stimulation based on a user input received at the controller.

21. The device of claim 20, wherein the user input includes at least one of the pulse duration, pulse waveform, pulse frequency, pulse pattern, voltage amplitude, and current amplitude of the electrical stimulation signal.

22. The device of any preceding claim, wherein the electrode is configured to be placed in contact with an outward facing surface of the tragus.

23. A system for modulating cerebral blood flow of a user, the system comprising:
the device of any preceding claim, the device further comprising a communication module connected to the controller of the device,
wherein the communication module is configured to send information from the device to an external computer system and to receive information from the external computer system, and the information received from the external computer system is used by the controller to determine the form of the electrical stimulation signal.

24. The system of claim 23 wherein information from the device received by the external computer system is compared to a secondary set of information stored on the external computer system to determine a set of actions to be performed by the device and/or the external computer.

25. A method for modulating cerebral blood flow of a user of any device according to any of claims 1 to 22, the method comprising:
producing, using a generator, an electrical stimulation signal;
determining, using a controller connected to the generator, the form of the electrical stimulation signal;
transmitting the electrical stimulation signal to an electrode, wherein the electrode is configured to be placed in contact with and provide the electrical stimulation signal to to the nerves innervating a tragus of the user,
wherein the electrical stimulation signal comprises a series of electrical pulses, each pulse repeats with a frequency of 1 Hz to 100 Hz and each pulse has a duration of 10 microseconds to 500 microseconds and an amplitude of 0.1 mA to 8 mA,
wherein the method is applied to the user at least once a day for 3 consecutive days.

26. The method of claim 25, wherein modulating cerebral blood flow is applied to the user for any of the following purposes: reducing systemic arterial blood pressure, reducing left ventricular hypertrophy, reducing pulmonary arterial blood pressure, treating heart failure and/or treating atrial fibrillation.

27. A method of modulating cerebral blood flow for the treatment of a disease or condition selected from the group consisting of hypertension, heart failure and/or atrial fibrillation, comprising administering to a subject an electrical stimulation signal using a device according to any one of claims 1 to 22, wherein the electrical stimulation signal comprises a series of electrical pulses, each pulse repeats with a frequency of 1 Hz to 100 Hz and each pulse has a duration of 10 microseconds to 500 microseconds and an amplitude of 0.1 mA to 8 mA, wherein the method is applied to the user at least once a day for 3 consecutive day.

28. The method or device of any preceding claim, wherein the electrode is configured to be placed in contact with an outward facing surface of the tragus and a reference electrode is configured to be placed in contact with an inward facing surface of the tragus.

29. The method of any of claims 25 to 28, wherein the electrical stimulation signal is transmitted to at least a first and second electrode, wherein the first electrode is configured to be placed in contact with the left tragus of the user and the second electrode is configured to be placed in contact with the right tragus of the user.

30. The method of claim 29, wherein the electrical stimulation signal applied to the left and right tragi is substantially the same electrical stimulation signal and is applied simultaneously or sequentially to each of the left and right tragi.

31. The method of claim 29, wherein the electrical stimulation signal applied to the left tragus is different to the electrical stimulation signal applied to the right tragus and the electrical stimulation signal applied to the left and right tragi is applied simultaneously or sequentially to each of the left and right tragi.

32. The method of claim 29, wherein the electrical stimulation signal applied to the left tragus is different to the electrical stimulation signal applied to the right tragus and the electrical stimulation signal applied to the left tragus is applied at a different time to the electrical stimulation signal applied to the right tragus.

33. The method or device of any preceding claim, wherein the electrical stimulation signal is of a sinusoidal, square, triangular, or “white noise” waveform.

34. The method or device of any preceding claim, wherein the electrical stimulation signal is a pulse waveform, the pulse being substantially a sinusoidal, square, triangular, or “white noise” waveform.

35. The method or device of any preceding claim, wherein the generated waveform is a symmetrical monophasic waveform, or a symmetrical biphasic waveform, or a symmetrical triphasic waveform.

36. The method or device of any preceding claim, where the generated waveform is an asymmetrical monophasic waveform, or an asymmetrical biphasic waveform, or an asymmetrical triphasic waveform.

37. The method of any of claims 25 to 36, wherein the method is applied to the user for a minimum of 5 minutes and a maximum of 2 hours per day.

38. The method of any of claims 25 to 37, wherein the method is applied to the user for a minimum of 5 minutes and a maximum of 2 hours per day and the method is applied at intervals separated by at least one day.

39. The method of any of claims 25 to 38, wherein
during a first period the method is applied to the user for between 5 minutes and 2 hours each day, the first period comprising a minimum of 3 consecutive days,
during a second period the method is stopped for at least 2 days, and
during a third period the method is applied to the user for between 5 minutes and 2 hours each day.

40. The method of any of claims 25 to 39, further comprising
measuring the user's blood pressure;
determining whether the user's blood pressure is greater than a predetermined threshold value;
if the user's blood pressure is greater than the predetermined threshold value, instructing the generator to produce the electrical stimulation signal.

41. The method of any of claims 25 to 40, wherein the determining, using a controller connected to the generator, the form of the electrical stimulation signal pulse comprises determining the pulse width, pulse frequency, pulse waveform and waveform pattern of the electrical stimulation signal.

42. A method of screening patients suitable for treatment using a device and a method of treatment according to any preceding claim the method comprising:
recording a patient's electrocardiogram for a minimum period of 1 minute;
analysing the power spectrum of heart rate variability;
determining the low frequency (LF) to high frequency ratio (HF) of heart rate variability spectrum (LF/HF);
determining whether the LF/HF ratio of heart rate variability spectrum is greater than a predetermined threshold value.

43. The method of claim 42, wherein the predetermined threshold value of LF/HF ratio of heart rate variability spectrum is 1.

44. A method of screening patients suitable for treatment using a device and a method of treatment according to any preceding claim the method comprising:
recording the baseline value of heart rate of a patient when resting in a supine position;
followed by recording the heart rate of the patient when actively standing in a vertical position, wherein the heart rate is recorded directly after the patient stands in the vertical position;
subsequently after a period of time recording the heart rate of the patient again while standing;
determining whether the difference between the heart rate value recorded immediately after standing and heart rate value recorded subsequently is less than a predetermined value.

45. The method of claim 44, wherein the predetermined threshold value of heart rate difference between the value recorded immediately after standing and heart rate value recorded between 10 and 20 s after standing is less than 6 (six) beats per minute.

46. The device or method of any of claims 1 to 41, wherein the earpiece comprises a stimulating electrode and a reference electrode; and the earpiece is configured such that when it is in position on the tragus, the stimulating electrode is in contact with a first face of the tragus and the reference electrode is in contact with a second, opposing face of the tragus.

47. The device or method of claim 46, wherein the controller selects the electrical stimulation signal such that over the course of a series of repeating pulses, a net conventional current flow is provided from the stimulating electrode to the reference electrode.

48. The device and method of any of claims 6 to 41, wherein the first electrode is a first stimulating electrode and the second electrode is a second stimulating electrode, and each earpiece comprises a stimulating electrode and a reference electrode wherein the electrical stimulation signal is applied between the stimulating electrode and the reference electrode.

49. A method of treating hypertension, left ventricular hypertrophy, heart failure and/or atrial fibrillation in a patient, the method comprising: (i) using a device according to any one of claims 1 to 22; and (ii) administering to the patient a pharmaceutically active composition for the treatment of hypertension, heart failure and/or atrial fibrillation.

50. A method as claimed in claim 49, wherein the pharmaceutically active composition comprises a beta-adrenoceptor antagonist.
